SEQUENCE LISTING (8 sequences)

SEQ ID NO: 1 - DNA, 5732 bp, Homo sapiens
  misc_feature (1)..(5732): Nucleotide sequence of human cannabinoid receptor 1
gcgccggcgccgcctcccgcacgctactccctctgccaccccttccttctccacttcttttccgcctccgcctcttcttgtctcccgcggcgccagcgccttcccttggcccgggcgggggcctcggctccctgcagagctctccgtagtcagtgggggatatttcgttctagcggacaaccagcccctgagctgggcgagaggtgccaagggagcttctgtcccgaggaccaggggatgcgaagggattgccccctgtgggtcactttctcagtcattttgagctcagcctaatcaaagactgaggttatgaagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggacattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctgtgagcctgatgcctccctggcagcacaggaaaagaatttttttttttaagctcaaaatctagaagagtctattgtctccttggttatatttttttaactttaccatgctcaatgaaaaggtgattgtcaccatgatcacttatcagtttgctaatgtttccatagtttaggtactcaaactccattctccaggggtttacagtgaagaaagcctgttgtttaagtgactgaacgatccttcaaagtctcaatgaaataggagggaaacctttggctacacaattggaagtctaagaacccatggaaaaatgccatcaaatgaataatgcctttgtaaccacaactttcactataatgtgaaatgtaactgtccgtagtatcagagatgtccatttttacaagttatagtactagagatattttgtaaaatgtattatgtcctgtgagatgtgtatcagtgtttatgtgctattaatatttgtttagttcagcaaaactgaaaggtagacttttatgagaacaatggacaagcagtggatacgtgtcaatgtgtgcactttttttctatattattgcccatgatataactttagaaataaaccttaatatttcttcaaatatctctatttaattttgacactgaaataaccgtaaaggtttatttttctgttacctcaacaagaagaatttgaagacttcaaaatattgagcagaattcattcatacttaaaaatttattagccctgcattttcataggaagacacattatcttctggactatagctgttctaatggattataatcagaatggaagagagaaagcatattgactttttttgagcgacatctctgactttctttagtctttagctattactggatctcttaagacagcatgtgttaatcttaatgtatatcgttatcactgtgcagttgctgtttacttgaatagtattgtgttcctatattccaggtttaagtagatttcatgcctgggtggccaaacaacagtcttcattttttttaattgaaaagaagtagtgtctggatcagtaaaattatactgtgtgtgagtgtgaatataaatgtgtgtatgtgtgtttctgtcctgtaactgttacagtaatgtcataaagtgagaaaactgtgaccaagtataaacttttaccacttgctgcactcttgcacatggattcagtttctaaaattgagttcttcctgtaatcttgttgataaaaatactgactccaaccattcaaaaatttcaccccatccctccttaagagattggatcaagtattactaaattgacctttaggtattacacaagaccagtgcttagcaaaaaataatgacaggcatccaaggaagggatgtatttgtagtgttattgccaggaaaggagagtactttggtttctgagcaccgaatattgagcaatatgtcagtcactaaaaggaagacagttctacagaaaaacaatggtaacatttttcaatagcgtgtgtagatagtatgcactatatacatcacgttaaagtaggactatcacacccagcccatgtggctaaaaaagctgaatcagacagtggatgagacacacaacggcagtgaagaaccgatacacttggcattgacgtctagctatgctgtatctgtgctttgcccacatgcccttggtgacagctgagcacccagctctgtcttggtaggtttgggctaaggaacaaatctctcctttgctcgtggttagcaagatacactcaagcatgaagataaacacagctgctttcttcttacaccccggtctcatgctccttaatggcgccatgggtgcttgttgggcctttttccagtaaggaatgatattgctgaagaatctacttaaccctgacaaattttaattataatctcttcttatacagataaaacatgactcctacaaggccccaaggtttacatagtctgaagtgaagtacagagctggcatctatctggtgatttctagctctcgagatacccaagcagcctgatggggcagttccccttcttacggttcacgctctaaggcaggatgtggcttatgagatactttgcattgtctgtctgcacaccttgaatctgcctgctggctcccttactttacctctctgtcatgtgcagatgaaggctcagggtgctagaggattagtaagatctctttctaaagacaggagagattatttacaagaagaactcaccagggtttagtttgcatttaagaattgccagtcttttgtcctgcatcatcttgaacattaatccacatgtttcagagctcaccaggcagtaccaatgctcttttcacagctatgaagagctagagaaattcttgttatggtagaaaaatttcacgattcatttttgaaactgcatttgtgcgtatgcagtgtagattttatagtgtgttgtgctttcaagatctaaatcatatataataaattaagggacaatggggctgacagcactaaacttggtgcttattgatattctaagaaatatctgtgaaatatcatcacgtatgttatacaaccttcatttaaaaaggtttaaaactagttagattcactttgacacttttcatatcatttcttaacccaagtgacgaaaacattgtccccaatgaatatactcattagaattaccatttgttaatatcactcattaattaaccccataattagatccattaatttaaatgatttaaatttaagtaagttttataaggtctgacatcagaggtatcttactttcctctgaggatgatgtacttgccctgaccatgcattttaccatcacacatgttcagaaagggccaaattcccaacctgctcattttttttttatcagagtcatgatgaatcagtcctagaatgtttcatttgcacaagtagggctgcctccaagaggaacctctgatttattttgtatgaaatatatgtgaaaggatatgaatctgagagatgctgtagacatctgtcctacacttgagatgatttccaagcctctctggcactttgagttaagtctatctggtattaaatgccaaggaccttttgctgcctaaatccactctgcaggaaataggcccaaccaccagatgagaattaggccctggatgagtagcgctatagttactgtcctgttgattaatttctgccatttcatgtccataaaagagaccacccatatcatgcacacaattagatttctcacactctaactgtatatttgtatgatattttaaaatctcctaaatgctgggcaatggctattaacaattaattgtcttgcactggccttctgatgaaatgttaacaatgcctattgtaatatagaaaaaaacattctatctactgatttgggctgaatgtatgtaaataggtttctaaaaagtcagatgtttgagcagtggcctacaaatcagtaattttcggatgggagagtttctttacattgccgtggcatcttaaaagctatcttcatgtaaattgactgtactaggcctactggggatcagagttcccaagaaaggaaaccttttcttgtatctggattcaaatttatttccaatgtttcaagcgggaaacatgactctttattgtctgtaaatctaacattattacttttcctcttagaagaatattgtattgttagatgtttgttgagctggtaacatcgttgcaaccactgcaatatcttcgttagtaatctgtataatactttgtatacaagtactggtaagattgttattaaatgtagcttcagtcattaaattactatagcaaagtagtacttcttctgtaatatttacaatgtattaagcccacagtatattttatttcaatgtaattaaactgttaacttattcaaagagaaaacatctcatcatgtctattgtccaaagttacctggaatcaaataaaaattctagattaccatgaagaacataaaaaaaaaaaaaaaa

SEQ ID NO: 2 - protein, 472 aa, Homo sapiens
  PEPTIDE (1)..(472): Amino acid sequence of human cannabinoid receptor 1
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 3 - DNA, 5387 bp, Homo sapiens
  misc_feature (1)..(5387): Nucleotide sequence of human cannabinoid receptor 2
gattgccccctgtgggtcactttctcagtcattttgagctcagcctaatcaaagactgaggttatgaagtcgatcctagatggccttgcagataccaccttccgcaccatcaccactgacctcctgggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggacattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctgtgagcctgatgcctccctggcagcacaggaaaagaatttttttttttaagctcaaaatctagaagagtctattgtctccttggttatatttttttaactttaccatgctcaatgaaaaggtgattgtcaccatgatcacttatcagtttgctaatgtttccatagtttaggtactcaaactccattctccaggggtttacagtgaagaaagcctgttgtttaagtgactgaacgatccttcaaagtctcaatgaaataggagggaaacctttggctacacaattggaagtctaagaacccatggaaaaatgccatcaaatgaataatgcctttgtaaccacaactttcactataatgtgaaatgtaactgtccgtagtatcagagatgtccatttttacaagttatagtactagagatattttgtaaaatgtattatgtcctgtgagatgtgtatcagtgtttatgtgctattaatatttgtttagttcagcaaaactgaaaggtagacttttatgagaacaatggacaagcagtggatacgtgtcaatgtgtgcactttttttctatattattgcccatgatataactttagaaataaaccttaatatttcttcaaatatctctatttaattttgacactgaaataaccgtaaaggtttatttttctgttacctcaacaagaagaatttgaagacttcaaaatattgagcagaattcattcatacttaaaaatttattagccctgcattttcataggaagacacattatcttctggactatagctgttctaatggattataatcagaatggaagagagaaagcatattgactttttttgagcgacatctctgactttctttagtctttagctattactggatctcttaagacagcatgtgttaatcttaatgtatatcgttatcactgtgcagttgctgtttacttgaatagtattgtgttcctatattccaggtttaagtagatttcatgcctgggtggccaaacaacagtcttcattttttttaattgaaaagaagtagtgtctggatcagtaaaattatactgtgtgtgagtgtgaatataaatgtgtgtatgtgtgtttctgtcctgtaactgttacagtaatgtcataaagtgagaaaactgtgaccaagtataaacttttaccacttgctgcactcttgcacatggattcagtttctaaaattgagttcttcctgtaatcttgttgataaaaatactgactccaaccattcaaaaatttcaccccatccctccttaagagattggatcaagtattactaaattgacctttaggtattacacaagaccagtgcttagcaaaaaataatgacaggcatccaaggaagggatgtatttgtagtgttattgccaggaaaggagagtactttggtttctgagcaccgaatattgagcaatatgtcagtcactaaaaggaagacagttctacagaaaaacaatggtaacatttttcaatagcgtgtgtagatagtatgcactatatacatcacgttaaagtaggactatcacacccagcccatgtggctaaaaaagctgaatcagacagtggatgagacacacaacggcagtgaagaaccgatacacttggcattgacgtctagctatgctgtatctgtgctttgcccacatgcccttggtgacagctgagcacccagctctgtcttggtaggtttgggctaaggaacaaatctctcctttgctcgtggttagcaagatacactcaagcatgaagataaacacagctgctttcttcttacaccccggtctcatgctccttaatggcgccatgggtgcttgttgggcctttttccagtaaggaatgatattgctgaagaatctacttaaccctgacaaattttaattataatctcttcttatacagataaaacatgactcctacaaggccccaaggtttacatagtctgaagtgaagtacagagctggcatctatctggtgatttctagctctcgagatacccaagcagcctgatggggcagttccccttcttacggttcacgctctaaggcaggatgtggcttatgagatactttgcattgtctgtctgcacaccttgaatctgcctgctggctcccttactttacctctctgtcatgtgcagatgaaggctcagggtgctagaggattagtaagatctctttctaaagacaggagagattatttacaagaagaactcaccagggtttagtttgcatttaagaattgccagtcttttgtcctgcatcatcttgaacattaatccacatgtttcagagctcaccaggcagtaccaatgctcttttcacagctatgaagagctagagaaattcttgttatggtagaaaaatttcacgattcatttttgaaactgcatttgtgcgtatgcagtgtagattttatagtgtgttgtgctttcaagatctaaatcatatataataaattaagggacaatggggctgacagcactaaacttggtgcttattgatattctaagaaatatctgtgaaatatcatcacgtatgttatacaaccttcatttaaaaaggtttaaaactagttagattcactttgacacttttcatatcatttcttaacccaagtgacgaaaacattgtccccaatgaatatactcattagaattaccatttgttaatatcactcattaattaaccccataattagatccattaatttaaatgatttaaatttaagtaagttttataaggtctgacatcagaggtatcttactttcctctgaggatgatgtacttgccctgaccatgcattttaccatcacacatgttcagaaagggccaaattcccaacctgctcattttttttttatcagagtcatgatgaatcagtcctagaatgtttcatttgcacaagtagggctgcctccaagaggaacctctgatttattttgtatgaaatatatgtgaaaggatatgaatctgagagatgctgtagacatctgtcctacacttgagatgatttccaagcctctctggcactttgagttaagtctatctggtattaaatgccaaggaccttttgctgcctaaatccactctgcaggaaataggcccaaccaccagatgagaattaggccctggatgagtagcgctatagttactgtcctgttgattaatttctgccatttcatgtccataaaagagaccacccatatcatgcacacaattagatttctcacactctaactgtatatttgtatgatattttaaaatctcctaaatgctgggcaatggctattaacaattaattgtcttgcactggccttctgatgaaatgttaacaatgcctattgtaatatagaaaaaaacattctatctactgatttgggctgaatgtatgtaaataggtttctaaaaagtcagatgtttgagcagtggcctacaaatcagtaattttcggatgggagagtttctttacattgccgtggcatcttaaaagctatcttcatgtaaattgactgtactaggcctactggggatcagagttcccaagaaaggaaaccttttcttgtatctggattcaaatttatttccaatgtttcaagcgggaaacatgactctttattgtctgtaaatctaacattattacttttcctcttagaagaatattgtattgttagatgtttgttgagctggtaacatcgttgcaaccactgcaatatcttcgttagtaatctgtataatactttgtatacaagtactggtaagattgttattaaatgtagcttcagtcattaaattactatagcaaagtagtacttcttctgtaatatttacaatgtattaagcccacagtatattttatttcaatgtaattaaactgttaacttattcaaagagaaaacatctcatcatgtctattgtccaaagttacctggaatcaaataaaaattctagattaccatgaagaacataaaaaaaaaaaaaaaa

SEQ ID NO: 4 - protein, 439 aa, Homo sapiens
  PEPTIDE (1)..(439): Amino acid sequence of human cannabinoid receptor 2
MKSILDGLADTTFRTITTDLLGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 5 - DNA, 4959 bp, Homo sapiens
  misc_feature (1)..(4959): Nucleotide sequence of human opioid receptor k1
agtgggagacgtgcgctgagaggcgggggctgcgctcggcggaacagcagccctcgggcggagagcggggccggggtccgagagcaggtgatgccaagagctgagcgggactcgtgagcgcgcggttcagcacctaccagggcgtcccgtaaaaaacctcgccttcgcctgtctctgggaaccatagcgccgcaggtgccgcctgtcctcgccttcctgctgcaatcgccccaccatggactccccgatccagatcttccgcggggagccgggccctacctgcgccccgagcgcctgcctgccccccaacagcagcgcctggtttcccggctgggccgagcccgacagcaacggcagcgccggctcggaggacgcgcagctggagcccgcgcacatctccccggccatcccggtcatcatcacggcggtctactccgtagtgttcgtcgtgggcttggtgggcaactcgctggtcatgttcgtgatcatccgatacacaaagatgaagacagcaaccaacatttacatatttaacctggctttggcagatgctttagttactacaaccatgccctttcagagtacggtctacttgatgaattcctggccttttggggatgtgctgtgcaagatagtaatttccattgattactacaacatgttcaccagcatcttcaccttgaccatgatgagcgtggaccgctacattgccgtgtgccaccccgtgaaggctttggacttccgcacacccttgaaggcaaagatcatcaatatctgcatctggctgctgtcgtcatctgttggcatctctgcaatagtccttggaggcaccaaagtcagggaagacgtcgatgtcattgagtgctccttgcagttcccagatgatgactactcctggtgggacctcttcatgaagatctgcgtcttcatctttgccttcgtgatccctgtcctcatcatcatcgtctgctacaccctgatgatcctgcgtctcaagagcgtccggctcctttctggctcccgagagaaagatcgcaacctgcgtaggatcaccagactggtcctggtggtggtggcagtcttcgtcgtctgctggactcccattcacatattcatcctggtggaggctctggggagcacctcccacagcacagctgctctctccagctattacttctgcatcgccttaggctataccaacagtagcctgaatcccattctctacgcctttcttgatgaaaacttcaagcggtgtttccgggacttctgctttccactgaagatgaggatggagcggcagagcactagcagagtccgaaatacagttcaggatcctgcttacctgagggacatcgatgggatgaataaaccagtatgactagtcgtggagatgtcttcgtacagttcttcgggaagagaggagttcaatgatctaggtttaactcagatcactactgcagtctgacatgaaaagatagaatttaaaataaacttttagagatctcatggtctgaagtcatcagatgcagaccacgttcgtgatcagtggaaacatcaaggacaaaaggtccacgcaacacatgcacctcttctctagggcacagaagcgaggcagagcctcagttcctctgatgaacaagggaacaggtcttttccttctggtttgctaggtaaggttcagcacccatctgctgtggccttcctatgaaacgtagtttcaaaagctcggtttaagaaaaaaggaaaagaaaaaacattgctttcagagctcaatgggcatccaatccagatgtttcacaatgacagcaacaacctccaacggaatgggaactatttccagggttcatgcccagctcactgtatgctgtgtggacatgcatttcatgtggctgtgtggtaagaattacagcttacatatggcttggacccacatctgaggaatctgatgttcacttataccagaacattatcttgctatttatgaaattatttaaacgttcaaaagattgtttttaacatggtttaatttcccaaaaactacagttttttttcttagcatgctattcaggtaaacagtcttataataaagcatgtcccattgtcaagaaacataaagtggtgtgaataccactgaaaatatatatatagtatcttctgtaaataatagtacctgtgtgaataaggaataggcttgcctcccagccaggcaatttcctgagggcacactaatgatatcccctgagttgctaagttgatgctgagacattttgctgggaattagtcatggcatgatctctttcagactccctgaataccatttagtcccgtaacagtgctcacaactcatgtgctaatgaatcacaaaggctttaactagctcccaggttgtagccttcgcaggatctagtttatttgccacatctctttatgaacatatagcgattcgcagatctctctattcacggagaggaaggtgttttgcttctgtagatctcaaggtactattttgtggctctcagcaggaagtagaattgttcctaaatgtgtgctgaatgaggacatgatgtccctcctggtgccaggacacatcctgcatggcattctgtgaagggcatacctgctgaggataatgccaggagcagcacatttagggtaattttgctaaaacttccagatgacatataaattccctctttttccccctgaaacttaccatttcagaggtaatggctttttctgtaatttgcctgagaagaaaaaagggataatttagaaataacactccaagcctatatattactagggctgcatcctctggaatttaatagaaaaattgaatatatagccctatgtaactacagacatcattacaactgttaagttctgttcaccatgcaagttctcggtggtgtatgtactctcaccttttggaaacaaaaccttgcattgaaggttttacacatggcttcagaatgttactctgaccatgtttgcctctactccagattatcatggcacctaacacagatgacatatggcagaatggaagtgatttctcctaatgaaaatgaagtgctcttttgagtatttcttgttttattgagattcctatctgcccttgtatttttataactgaatgtaaataggaattgcttaaatactggtttacactaatcccatagttaaaaatcttggctatggtacaggaaggatatctatatagaggatagttccagaaacaagccctggtgaaataggaaatataaaaacttctttatggaacaggaaaacccaagagactttctcgtcagattcctttccagtgcaaagtataacagagataggattatgagatgaaatcacagtagtgtgtttcctttaattttttctgtcttctttttttttttttttttttttgagacggagtctcgctctgttgcccaggctggagtgcggtagcacagtcttggctcactgcaacctccacctcctgggttcaagcgattctcctgcctctgcctcctaagtagctgggattacaggcgtgcaccgccaagcccggctaatttttgtatttttagtagagacggggtttcaccatgttatgcaggctggtctcaaactcctgacctcagaatattcacctgcctcggcctcccaaagtgctgggattacagatgtgaatggccacgcctggcctgtttcctttaagttagcacttcacgtgctcttacagcgttaattgccattattggtttgcctgattttcctgcatctcctcatatgtaagtctgcagcccaaggagagaatagtagctgtatgtgcccacaaggggtgtccaagcttcttgattcttgttaatatcgactccccaacattgaaaaaggagtgaagaagggaatatcattttagggtactgctttaaaggaatgataaagtaaacaacgtagcagggaaaataccacttgcacaaatgtatttctttgtcaaagtgtgtacacgctttctgtttgtcctgatgtcctgctgtgaagcagggaccacattgccatttattcattgttccaggcaatgcatttgtccaaacccctctggctctctgatatctctcatttcttgatgttcctattctctgacctcaacaaggaggacactgtctgttgtgtctctttgagactagaactccatgtgtttaaccttcagatccattctctacatgaaccctattctttccaccctggtaagaaggtctggaagacaggtcctaacgaggggtcagagaaggcacagagaaatgaaccagccatgcaggggcagcactgaggaggaaaccccccactctaactgggaggagagtggaaacactcttagtatctcagagtagttaccaatctaagagcatccttcgagtttaagtcaaaactctaggcttaataccaaaaataatacatcatttggaaaaatagcttggagaaaaaaaatcaataaagagtcacataaaatcttaagaaaatggttctattctgtgatatattactaagaaatacagttcttaaacgtgtataactattgtcagacaatttataggtgtttcatctagtcctgggatgaaatacgatgactcatgcaaattcagggagcacactggctgactttaaattggaacattgtaaagtgaaagctgtgaaaggttgtgtgtcctatcaatgagatttattttctaaccaagagctgttcggatgattcagtataaatacgaaaatcacatccaaaaacggtataacccagcttccttaaggcaattttcttctctgaaacaagaatatactcatatgttctttactataatgtatatgatttttatcttgtactttaaaagatgataattatgcattgtatatacgattgtgtgccttgcaataaaatcaaaactgtacctgctgaaaatcacaacagtc

SEQ ID NO: 6 - protein, 380 aa, Homo sapiens
  PEPTIDE (1)..(380): Amino acid sequence of human opioid receptor k1
MDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQSTSRVRNTVQDPAYLRDIDGMNKPV

SEQ ID NO: 7 - DNA, 2178 bp, Homo sapiens
  misc_feature (1)..(2178): Nucleotide sequence of human opioid receptor 1; transcript variant MOR-1
gatgagcctctgtgaactactaaggtgggagggggctatacgcagaggagaatgtcagatgctcagctcggtcccctccgcctgacgctcctctctgtctcagccaggactggtttctgtaagaaacagcaggagctgtggcagcggcgaaaggaagcggctgaggcgcttggaacccgaaaagtctcggtgctcctggctacctcgcacagcggtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccctaacagggtctcatgccattccgaccttcaccaagcttagaagccaccatgtatgtggaagcaggttgcttcaagaatgtgtaggaggctctaattctctaggaaagtgcctgcttttaggtcatccaacctctttcctctctggccactctgctctgcacattagagggacagccaaaagtaagtggagcatttggaaggaaaggaatataccacaccgaggagtccagtttgtgcaagacacccagtggaaccaaaacccatcgtggtatgtgaattgaagtcatcataaaaggtgacccttctgtctgtaagattttattttcaagcaaatatttatgacctcaacaaagaagaaccatcttttgttaagttcaccgtagtaacacataaagtaaatgctacctctgatcaaagcaccttgaatggaaggtccgagtctttttagtgttttgcaagggaatgaatccattattctattttagacttttaacttcaccttaaaattagcatctggctaaggcatcattttcacctccatttcttggttttgtattgtttaaaaaaataacatctctttcatctagctccataattgcaagggaagagattagcatgaaaggtaatctgaaacacagtcatgtgtcagctgtagaaaggttgattctcatgcactgcaaatacttccaaagagtcatcatgggggatttttcattcttaggctttcagtggtttgttcct

SEQ ID NO: 8 - protein, 400 aa, Homo sapiens
  PEPTIDE (1)..(400): Amino acid sequence of human opioid receptor 1; transcript variant MOR-1
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

(19) United States
(12) Patent Application Publication
(10) Pub. No.: US 2015/0268257 A1
(43) Pub. Date: Sep. 24, 2015

(54) METHOD AND DEVICE FOR DETECTING ILLEGAL DRUGS

(71) Applicant: SECURETEC DETEKTIONS-SYSTEME AG, Neubiberg (DE)

(72) Inventors: Sebastian Klaus, Neuried (DE); Frank Schwieger, Taufkirchen (DE); Verena Zimmermann, Muenchen (DE)

(73) Assignee: SECURETEC DETEKTIONS-SYSTEME AG, Neubiberg (DE)

(21) Appl. No.: 14/436,952

(22) PCT Filed: Oct. 21, 2013
(86) PCT No.: PCT/EP2013/071961

§ 371 Date: Apr. 20, 2015

(30) Foreign Application Priority Data
Oct. 22, 2012 (EP) 12189463.8

(51) Int. Cl. G01N 33/94

(52) U.S. Cl. CPC G01N 33/9486; G01N 33/948; G01N 2333/726

(57) ABSTRACT

The present invention relates to a method for determining analytes, in particular illegal drugs, in a sample and also to test elements, sampling elements and kits suitable for carrying out the method.

The present invention relates to a method for determining analytes, in particular illegal drugs, in a sample and to test elements, sampling elements and kits suitable for carrying out the method.

Immunoassays which are based, for example, on “lateral flow technology” are rapid test systems which are recognised and used worldwide for detecting analytes in body fluids or on surfaces. In tests of this type, a sample containing the analyte is usually taken up from a surface using a suitable sampling element and is transferred to the test element (for example to a test strip). The analyte is then detected in the test element by means of an immunological detection reaction which is based on the formation of immunocomplexes of antigens and antibodies.

In general, immunological detection systems provide a high specificity and sensitivity due to the biophysical and biochemical characteristics of the antigen-antibody bond. This is very important especially in the detection of illegal drugs (for example amphetamines, methamphetamines, cannabis, cocaine, heroin). Here, on the one hand there is a need to rapidly detect the use of illegal drugs. On the other hand, the test formats should also have a high sensitivity and specificity to rule out false-positive and false-negative test results and to provide reliable information about which drug has been used.

Since 2008, mixtures of various herbs and aromatic substances have been available which, according to the information on the packaging, are intended to be used for burning in rooms and “fragrancing” them. However, the fact is that these herbal mixtures are used as intoxicating drug in the same way as cannabis, in a direct manner or combined with tobacco by inhaling the occurring smoke. As the result of professional marketing, a strong presence in the mass media and the general, but incorrect, assumption that these are legally available herbal mixtures with a “cannabis-like” intoxicating effect known as “legal highs”, products such as “Spice”, “Smoke”, “Lava Red” and a large number of derived products have become very popular.

However, chemical analyses show that the pharmacological effect of such herbal mixtures is to be attributed to synthetic cannabinoids which are impregnated on the herb leaves and that the herb leaves merely act as carrier material. Thus, for example it has been known since 2008 that undeclared synthetic cannabinoids such as JWH-018 and CP-47497 are responsible for the effect of “Spice”. Synthetic cannabinoids are a group of more than 500 substances, most of which were produced in the 1980s and 1990s as possible receptor agonists for the human cannabinoid receptors. The objective was to develop new analgesically effective medicaments without the psychoactive component of cannabis.

In addition to the existing addiction potential, the use of synthetic cannabinoids is associated with a high health risk (particularly during long-term use), since there are no reliable data in respect of dose-effect relationships, undesirable side effects or toxicology. Users of products which contained synthetic cannabinoids reported a cannabis-like effect including the undesirable side effects usual in cannabis use, such as dizziness, increased anxiety, palpitations and drowsiness (Vandrey et. al., Drug Alcohol Depend. (2012), 120, 238-241).

Apart from the mostly unknown health risks for users, there are also risks to the general public. This is the case, for example, when people are under the influence of synthetic cannabinoids in the workplace or when participating in road traffic, as a result of which safety risks are presented which are comparable with the use of other illegal drugs, such as amphetamines, benzodiazepines, ketamines, cocaine, methamphetamines, cannabis and opioids. This led to a strict prohibition of these substances or of products containing these substances in many countries, inter alia in large parts of Europe.

In spite of this ban, products containing synthetic cannabinoids continue to be sold, particularly over the Internet. Accordingly, it is crucial to have sensitive and reliable rapid drug tests for detecting synthetic cannabinoids particularly in road traffic checks by the police and customs, in the detection of drug smuggling, or in workplace checks. However, synthetic cannabinoids cannot be detected or cannot be detected satisfactorily using the presently available rapid drug tests, because current rapid drug tests do not detect this substance class, or only detect it sometimes.

EP 0 699 906 A2 discloses a rapid drug test which is commercially available under the name of DrugWipe® (Securetec Detektions-Systeme AG) as a surface, sweat or saliva test. The test uses a substantially plastics wiping element (“wiper”) with a non-woven fabric welded thereon, by which a sample of the analyte is taken (for example from a surface or out of a solution) and is then transferred directly onto an immunochromatographic test strip (“lateral flow” technology) which is stored in a single-use housing.

By bringing the test strip into contact with an aqueous solution (water or buffer with different reagents), chromatography is initiated, wherein the result of the determination can be read visually or using a suitable measuring device. The analyte is detected in that antibodies bound to drug molecules (antigens) bind to a test line and due to their gold label, they form a coloured line which can be detected optically in the read-out window. Antibodies which are not loaded with drug molecules are intercepted before they reach the test line by polyhaptens which are present on the test strip in immobilised form, and are not optically visible in the read-out window.

Compared to all other commercially available rapid drug tests, DrugWipe® is the only product which forms a coloured line when a specific analyte is present in a sample and, in this respect, it has a so-called positive indication. All other commercial products have a negative indication whereby the non-appearance of a line is evaluated as positive evidence of the respective analyte. A further advantage of DrugWipe® is the small sample volume required for detecting drugs and other substances. Whereas for DrugWipe® a sample volume of less than 25 μl is sufficient, other commercial tests require a sample volume of at least 100 μl up to several millilitres.

The low sample volume required for carrying out the DrugWipe® rapid drug test affords a significant advantage over other commercially available test systems, because drug users often have a very dry mouth due to the physiological effect of the drugs and only a small amount of saliva is available for sampling. This is an advantage particularly in the case of users of cannabis and synthetic cannabinoids, since the use of these drugs is always accompanied by a very dry oral cavity with little saliva. Consequently, precisely in these cases, often only very small sample volumes are available for which, with the exception of DrugWipe®, rapid drug tests are generally inadequate for obtaining a reliable and accurate result. If it is even possible to carry out a rapid drug test, sampling often takes several minutes which is unreasonable both for the person providing the sample and for the person taking the sample.

The rapid test OraLab® 6 developed by Varian is used to detect drugs from samples of saliva and is also based on lateral flow technology. Whereas the specificity of the test is approximately 90-100%, the sensitivity in the case of amphetamines and opiates is only between 50 and 90%, in the case of Δ⁹-tetrahydrocannabinol and cocaine it is sometimes even significantly less than 50% (see DRUID study, published at TIAFT 2009 in Geneva). Furthermore, a serious disadvantage of this test is that relatively large volumes of sample are required which, in the case of chronic drug users, are often unavailable.

The rapid drug test DrugTest® 5000 manufactured by Drager which is also based on lateral flow technology provides a reliable testing system for drug detection according to the data produced by the DRUID study (TIAFT 2009, Geneva). However, this test format has two significant disadvantages. Firstly, the results on the test strip can only be read using a read-out device which is very expensive to produce and is only partly suitable for tough field use in the open air. Secondly, for this test, 300 μl of saliva are required as the sample volume, which is why sampling, particularly in the case of drug users who have a dry mouth, sometimes lasts several minutes. In this respect, considerable problems arise for this test with regard to economy and simple handling ability.

The Rapid STAT® test kit supplied by Mavand is a further commercially available rapid drug test in which a puffer-diluted sample of saliva is incubated on an immunochromatographic test strip having labeled binding partners and which, according to manufacturer's information, allows a detection as far as a lower limit of 15 ng/ml of Δ⁹-tetrahydrocannabinol. A significant disadvantage of this test is the extremely complex and laborious handling thereof which makes it unsuitable for use by traffic police, and also the comparatively low specificity for Δ⁹-tetrahydrocannabinol of 80-90% (see DRUID study, TIAFT 2009, Geneva). According to a study carried out at the University of Mainz, the rate of false-positive test results for Δ⁹-tetrahydrocannabinol is more than 10% and the overall specificity is 84 (published at GTFCH 2009, Mosbach, http://www.gtfch.org/cms.images/stories/media/tk/tk76_{—}2/abstractsposter.pdf).

WO 2005/075982 A2, EP 0 811 842 A2 and EP 0 699 906 A2 disclose methods for determining analytes in a body fluid or on a contaminated surface. To carry out the method, a test kit is used each time which comprises a test strip of one or more capillary-active materials capable of chromatography, a sampling element separate from the surface of the test strip, and a pressing device for bringing the surface of the test strip into contact with the sampling element. Here, at least a part of the sample is transferred from the sampling element onto the test element and the analyte is analysed after chromatography has started and after binding to an analyte-specific binding partner has occurred.

WO 2005/121793 A2 discloses a method for detecting a methamphetamine in a liquid sample. For carrying out the method, a kit is preferably used which comprises a chromatographic test strip and optionally a sampling element. For its part, the test strip comprises (a) a dry porous material on which a pseudoephedrine/carrier conjugate or an antibody which can bind both to the methamphetamine and to the conjugate is immobilised in a detection zone, and (b) a separate label release zone which can release into the liquid either the antibody in labeled form, if the detection zone contains immobilised pseudoephedrine/carrier conjugate, or detectable pseudoephedrine/carrier conjugate, if the detection zone contains immobilised antibody.

A disadvantage of the detection methods described in EP 0 699 906 A2, EP 0 811 842 A2, WO 2005/075982 A2 and in WO 2005/121793 A2 is furthermore that before the start of chromatography, the sample is not pretreated and the analyte-specific binding partner is not incubated directly with the analyte. Instead, the analyte and the analyte-specific binding partner only come into contact with one another during the course of chromatography, as a result of which in particular the sensitivity of the method is adversely affected and the legally required minimum detection limits for drug molecules are at least sometimes not achieved.

Furthermore, the methods described in WO 2005/075982 A2, EP 0 811 842 A2, EP 0 699 906 A2 and in WO 2005/121793 A2 have the disadvantage that the analyte-specific binding partner is an antibody in each case. This is a problem insofar as different drug molecules, such as Δ⁹-tetrahydrocannabinol and very diverse synthetic cannabinoids cannot usually be detected by a single antibody, because antibodies usually have a restricted selectivity and do not allow a wide-band detection of substances with a different chemical structure. If Δ⁹-tetrahydrocannabinol and very diverse synthetic cannabinoids are to be simultaneously determined in a single test, several hundred antibodies would possibly have to be developed, which is not viable either technically or economically.

The reason why conventional, antibody-based rapid drug tests do not generally allow the determination of synthetic cannabinoids is because synthetic cannabinoids comprise a large number of substances which, for their part, have a high structural variability (i.e. they only have slight structural similarities among one another or none at all). Furthermore, there is not usually a pronounced structural similarity to the natural cannabis active ingredient Δ⁹-tetrahydrocannabinol, as is clear for example from FIG. 1. Thus, the detection of synthetic cannabinoids usually requires a complex, apparatus-based laboratory examination which cannot be carried out promptly or locally.

The only presently available rapid test for synthetic cannabinoids is provided by the company DrugCheck (http://www.drugcheck.com/dc_info-k2-spice.html), although only the substances JWH-018 and JWH-073 (see FIG. 1) can be detected. Moreover, this rapid test is a urine test which is used reluctantly in practice due to the complex sampling procedure and it also only detects metabolites of the substances to be determined (and not the actual psychoactive substances). Furthermore, the metabolites can sometimes be detected several weeks after actual use, although the user is not experiencing any psychoactive impairment at that time.

Taking these facts into account, the object of the present invention was thus to provide a method for determining analytes, in particular for determining illegal drugs, in which method at least some of the disadvantages of the prior art are overcome. In particular, the method should allow a simple, fast and reliable onsite determination of a large number of cannabinoids and opioids, specifically of Δ⁹-tetrahydrocannabinol and synthetic cannabinoids, which determination can be carried out using a small sample volume and at the same time ensures a high sensitivity and specificity.

This object is achieved according to the invention by a method for determining an analyte in a sample, comprising the steps of:

(a) providing a test element,

(b) applying the sample to the test element, and

(c) determining the presence or/and quantity of analyte on the test element,

wherein the receptor molecule comprises the ligand-binding domain of a narcotic-binding receptor molecule, in particular the ligand-binding domain of a cannabinoid-binding or opioid-binding receptor molecule, and

wherein the ligand-binding domain of the receptor molecule is present in a native, shortened or mutated form and optionally is conjugated with a heterologous molecule.

The first step of the method according to the invention requires the provision of a test element which is suitable for determining the analyte and which comprises at least one receptor molecule binding the analyte. The test elements used for this purpose fundamentally comprise any physical form which is familiar to a person skilled in the art and is suitable for determining the presence or/and quantity of an analyte in a sample. The test element is preferably configured such that, when the analyte to be determined is present, it generates an optically detectable signal which allows a qualitative or/and quantitative determination of the analyte.

Examples of test elements within the context of the present invention include in particular test elements for carrying out a heterogeneous, homogeneous or chromatographic test, an ELISA (enzyme-linked immunosorbent assay) or a FRET assay (fluorescence resonance energy transfer). Test elements of this type are known in the specialist field and can be specifically selected by a person skilled in the art according to respective requirements. If desired or required, the test elements can contain microfluidic structures, such as microducts, steps, branches or/and chambers, as a result of which an improved transfer of the sample in the test element can possibly be achieved. In a preferred variant of the invention, the test element is a chromatographic test strip, to which the analyte can be applied, for example as an aqueous or non-aqueous solution.

In a variant of the invention, the chromatographic test strip is formed from a single, optionally strip-shaped material which is capable of chromatography. However, the chromatographic test strip preferably comprises a plurality of capillary-active faces, arranged overlapping on a carrier layer, which consist of the same or different chromatographic materials which are in fluidic communication with one another and in this manner form a transportation path, along which a fluid, driven by capillary force, can flow through all regions of the test element. In this respect, any known liquid-absorbing, porous or capillary-active material, such as cellulose and derivatives thereof, glass fibres, as well as non-woven fabric and woven fabric consisting of synthetic or natural materials, can be used as the chromatographic material. Chromatographic test strips which can be used within the scope of the present invention are described, for example, in EP 0 699 906 A2; reference is hereby explicitly made to the disclosure thereof.

The analyte-binding receptor molecule which can be present on the test element in soluble form or in immobilised form comprises, according to definition, a ligand-binding domain which is optionally conjugated with a heterologous molecule. The expression “ligand-binding domain” as used in the present application denotes a sequence of several, i.e. at least two, successive amino acids of a receptor molecule, via which an analyte to be determined binds to the receptor molecule. Accordingly, in particular a polypeptide or protein which is preferably derived from a membrane protein, more preferably from a transmembrane protein, most preferably from a G-protein-coupled receptor, is used as the analyte-binding receptor molecule. If the ligand-binding domain is conjugated with a heterologous molecule, the heterologous molecule is preferably a heterologous polypeptide, more preferably an immunoglobulin domain.

The analyte-binding receptor molecule preferably comprises the ligand-binding domain, in particular the extracellular ligand-binding domain of a narcotic-binding receptor molecule, such as a cannabinoid-binding or opioid-binding receptor molecule. More preferably, within the scope of the present invention, a receptor molecule is used which comprises the ligand-binding domain, in particular the extracellular ligand-binding domain of a receptor molecule selected from the group consisting of cannabinoid receptor 1, cannabinoid receptor 2, opioid receptor δ, opioid receptor κ, opioid receptor μ1 and opioid receptor μ2.

According to the invention, the ligand-binding domain can be a native ligand-binding domain, a shortened form of a native ligand-binding domain or a mutated form of a native ligand-binding domain, the expression “native ligand-binding domain” denoting the ligand-binding domain of a naturally occurring receptor molecule and the naturally occurring receptor molecule preferably originating from a mammal, preferably from a human being. Naturally occurring receptor molecules more preferably used within the scope of the present invention comprise human cannabinoid receptor 1 (see FIGS. 6 and 7), human cannabinoid receptor 2 (see FIGS. 8 and 9), human opioid receptor κ (see FIGS. 10 and 11), human opioid receptor μ1 and human opioid receptor μ2.

Within the scope of the method described herein, an analyte-binding receptor molecule can preferably be used which has a shortened amino acid sequence compared to a naturally occurring receptor molecule or/and which comprises a shortened form of a native ligand-binding domain. The expression “shortened form of a native ligand-binding domain”, as used in the present application, denotes any fragment of a native ligand-binding domain which is capable of binding the analyte and which, compared to the ligand-binding domain of a naturally occurring receptor molecule, has a shortened amino acid sequence, for example an amino acid sequence shortened by between 5% and 75%, it being possible for the shortening of the amino acid sequence to be realised at the N-terminus, at the C-terminus or/and between the N-terminus and the C-terminus of the naturally occurring receptor molecule or of the native ligand-binding domain of the receptor molecule.

If the analyte-binding receptor molecule has a shortened amino acid sequence, then it is preferred according to the invention that the amino acid sequence is shortened by up to 10%, 20%, 30%, 40% or 50% compared to the amino acid sequence of the native analyte-binding receptor molecule. However, it has proved particularly preferable to use an analyte-binding receptor molecule in which the amino acid sequence of the ligand-binding domain is shortened by up to 10%, 20%, 30%, 40% or 50% compared to the amino acid sequence of the ligand-binding domain of a naturally occurring receptor molecule, such as the amino acid sequence of the ligand-binding domain of human cannabinoid receptor 1, human cannabinoid receptor 2, human opioid receptor κ, human opioid receptor μ1 or human opioid receptor μ2.

Furthermore, in the method described herein, in particular an analyte-binding receptor molecule can be used which has a mutated amino acid sequence compared to a naturally occurring receptor molecule or/and which comprises a mutated form of a native ligand-binding domain. The expression “mutated form of a native ligand-binding domain”, as used in the present application, denotes a genetically altered variant of a native ligand-binding domain which is capable of binding the analyte and which, compared to the ligand-binding domain of a naturally occurring receptor molecule, has an altered amino acid sequence, for example an amino acid sequence with a sequence identity of between 50 and 99%, it being possible for the alteration in the amino acid sequence to be realised at the N-terminus, at the C-terminus or/and between the N-terminus and the C-terminus of the naturally occurring receptor molecule or of the native ligand-binding domain of the receptor molecule.

The mutations can be of a natural origin (for example point mutations or transcript or splice variants of a naturally occurring gene) or can be introduced using recombinant methods known in the specialist field, for example by site-specific deletion, exchange or/and insertion of nucleotides on DNA level or of amino acids on protein level. In this manner, at least one amino acid exchange results within the amino acid sequence of the naturally occurring receptor molecule, particularly within the amino acid sequence of the native ligand-binding domain, as a result of which it is possible, for example, to achieve an increase in the thermal or/and chemical stability of the receptor molecule. The objective is to maintain the binding characteristics of the native receptor molecule with respect to the analyte and at the same time to increase the stability thereof for use in a test element, in which case specifically a stabilisation in connection with the processes of drying (dehydrating), storing for 1-2 years in a dry state and rehydration is of interest.

If the analyte-binding receptor molecule has a mutated amino acid sequence, it is preferred that this amino acid sequence is identical to at least 80%, in particular to at least 95%, to the amino acid sequence of the native analyte-binding receptor molecule. Particularly preferred in this connection is the use of an analyte-binding receptor molecule in which the amino acid sequence of the ligand-binding domain is identical to at least 80%, in particular to at least 95%, to the amino acid sequence of the ligand-binding domain of a naturally occurring receptor molecule, such as the amino acid sequence of the ligand-binding domain of human cannabinoid receptor 1, human cannabinoid receptor 2, human opioid receptor κ, human opioid receptor μ1 or human opioid receptor μ2.

Specific examples of receptor molecules or of ligand-binding domains which can be used within the scope of the method according to the invention include, in addition to the cannabinoid receptors and opioid receptors shown in FIG. 6-13 of the present application, inter alia the natural or recombinantly produced DNA sequences and amino acid sequences described in WO 92/02640 A1, WO 95/07983 A1, WO 98/33937 A2, WO 00/04046 A2, WO 03/002718 A2, WO 2004/007551 A1, U.S. Pat. No. 6,235,496 B1 and US 2002/0077285 A1. Reference is hereby explicitly made to the disclosure of the documents mentioned above.

A significant advantage of the method according to the invention is that the analytes can be detected with a high degree of specificity and sensitivity. In particular, however, using a single receptor molecule, the specific and sensitive detection of structurally very different analytes from a specific substance class can be carried out. Thus, for example it is possible to detect both Δ⁹-tetrahydrocannabinol and all presently known as well as future synthetic cannabinoids using the ligand-binding domains of human cannabinoid receptor 1 and human cannabinoid receptor 2.

The reason for this is that a substance is classified as an illegal cannabinoid when it develops a psychoactive effect comparable to Δ⁹-tetrahydrocannabinol in humans. However, this can only be the case when, following use, the substance induces the signal transduction pathways known for cannabinoids by binding to one human or to both human cannabinoid receptors. Equally, it is thus possible to detect structurally different molecules from the substance class of opioids, such as codeine, desomorphine, fentanyl, heroin, methadone and morphine by means of the ligand-binding domains of human opioid receptors (for example opioid receptor μ1 and opioid receptor μ2).

To facilitate the detection of the analyte using the test element described herein, the analyte-binding receptor molecule can comprise a detectable label, such as an enzyme label, a dye label, a fluorescence label or a particle label. Within the scope of the present invention, a particle label in which the analyte-binding receptor molecule is bound covalently or non-covalently to the surface of suitable nanoparticles (for example gold or platinum particles) has proved to be advantageous. Particularly preferably, the label is a gold label which has the advantage that the user can directly detect the test result in an optical-visual manner and evaluate it. Techniques, whereby labels described above can be introduced into a molecule to be labeled, are known to a person skilled in the art and in this respect will not be described further.

In a further step of the method according to the invention, to determine the analyte using the test elements described herein, a sample containing the analyte is applied to the test element using suitable means. In a preferred variant, the sample is taken up from a surface by a sampling element which can have one or more sampling faces and the sampling element is then brought into contact with the test element, at least a part of the sample being transferred from the sampling element to the test element. In principle, sampling can be carried out in any manner, for example by scratching, wiping or suctioning the sample from a suitable surface, in particular from a surface of the body, such as for example the tongue or skin.

The sampling element is preferably a wiping element which allows a sample to be wiped from a surface to be examined and allows the sample to then be transferred onto the test element, for example by making use of the capillary effects. The wiping element has one or more (mutually independent) wiping faces; in the case of a plurality of wiping faces, wiping elements with 2, 3 or 4 wiping faces are preferred. If a wiping element having a plurality of wiping faces is used, a plurality of (mutually independent) test elements is usually also used, each of these test elements being respectively brought into contact with a wiping face of the wiping element.

The at least one wiping face which is preferably welded to a surface of the wiping element can fundamentally consist of any material which a person skilled in the art deems to be useful for the purposes of the present invention and which does not adversely affect a subsequent transfer of the analyte onto the test element. Here, absorbent materials, in particular woven fabrics, non-woven fabrics, or/and porous matrices (for example membranes and sponges) have proved to be specifically expedient. Particularly preferably, within the scope of the invention, non-wovens, in particular non-wovens based on cellulose fibres, polyester fibres or/and glass fibres are used, it being possible for the fibres to be held together, if required, by an organic binder. Suitable non-wovens and fibres are described, for example, in DE 38 02 366 A1 and in EP 0 699 906 A2; reference is explicitly made to the disclosure thereof.

As far as the external form of the wiping surface(s) is concerned, there are no fundamental restrictions in respect of thickness, dimensions and shape. The thickness of the wiping face(s) or of the material used for this is of minor importance for the purposes of the present invention and is usually within a range of 0.1 to 3 mm. The dimension of the wiping face(s) is advantageously adapted to the dimension of the test element, i.e. the width of the wiping face(s) should neither exceed nor fall below the width of the test element. Preferred dimensions of the wiping surface(s) with regard to the length are within a range of 0.3 to 2 cm, and with regard to the width, within a range of 0.3 to 1 cm.

The shape of the wiping face(s) can be adapted to the particular requirements imposed on the surface to be examined, a triangular, quadrangular (for example square, rectangular, diamond-shaped) or roll-shaped configuration of the wiping face(s) being considered particularly advantageous. The advantage of a roll-shaped configuration of the wiping face(s) is that the large surface of the wiping face(s) provides a particularly good contact between the wiping element and the test element and thus an increase in the sensitivity of the method can be achieved.

To ensure a particularly high sensitivity and specificity during the determination of the analyte, the test element or/and the sampling element comprises an analyte transfer reagent. The analyte transfer reagent, which promotes the transfer of the analyte from the surface to be examined to the sampling element or/and promotes the subsequent transfer of analyte from sampling element to the test element in particular by blocking free binding sites on the sampling element or/and promotes the influencing of the analyte characteristics, can be impregnated for this purpose, for example on the test element or/and on the sampling element, in particular in the region of optionally present sampling face(s).

In a variant of the invention, the test element used for determining the analyte comprises the analyte transfer reagent described above, while in another embodiment, the sampling element contains the analyte transfer reagent. Particularly preferably however, both the test element and the sampling element comprise an analyte transfer reagent which contains in each case at least one analyte-non-specific substance selected from the group consisting of a protein, a protein mixture, a carbohydrate and a sugar alcohol. The concentration of analyte-non-specific substance in the analyte transfer reagent can be adapted according to the particular requirements imposed on the test element by a person skilled in the art, but is usually approximately 0.01 to approximately 15 wt.-%, based on the total weight of the analyte transfer reagent.

If the test element comprises the analyte transfer reagent, it is also considered preferable for the analyte transfer reagent to contain an analyte-non-specific protein or/and an analyte-non-specific protein mixture, in particular an analyte-non-specific protein. Preferred according to the invention as protein is a substance selected from the group consisting of gelatine, ovalbumin and bovine serum albumin, while it is possible to use as the protein mixture skimmed milk powder, for example. On the other hand, the sampling element preferably comprises an analyte transfer reagent which contains a carbohydrate or/and a sugar alcohol, in particular a sugar alcohol.

The term “carbohydrate” as used in the present application denotes monosaccharides and oligosaccharides of the general molecular formula CₙH₂ₙOₙ which can in each case be of a natural or synthetic origin. The following are used in particular as monosaccharides: naturally occurring tetroses, pentoses and hexoses, such as erythrose, threose, ribose, arabinose, lyxose, xylose, allose, altrose, galactose, glucose, gulose, idose, mannose, talose and fructose which can in each case be in the D-form or L-form. It is possible to use as oligosaccharides in particular naturally occurring disaccharides and trisaccharides, such as lactose, maltose, saccharose, trehalose, gentianose, kestose and raffinose. In a particularly preferred embodiment of the invention, the carbohydrate is a substance selected from the group consisting of glucose, lactose, maltose, mannose and saccharose.

The term “sugar alcohol” as used in the present application denotes monosaccharide sugar alcohols of the general molecular formula CₙH₂ₙ₊₂Oₙ and disaccharide alcohols of the general molecular formula CₙH₂ₙOₙ₋₁ which can in each case be of a natural or synthetic origin. Preferred monosaccharide sugar alcohols include glycerol, erythritol, threitol, ribitol, arabinitol, xylitol, allitol, altritol, galactitol, glucitol, iditol and mannitol which can in each case be in the D-form or L-form. It is possible to use as disaccharide sugar alcohols in particular isomalt, lactitol and maltitol. In a particularly preferred embodiment of the invention, the sugar alcohol is a substance selected from the group consisting of glucitol, glycerol, lactitol, mannitol and xylitol.

After taking up the sample, the sampling element can be brought into contact with a region of the test element, preferably by light mechanical pressing on the sampling face(s) thereof, which region is configured for applying the sample containing the analyte, wherein at least a part of the sample is transferred from the sampling element onto the test element. The pressure at which the sampling element is pressed onto the test element should be at least great enough for the surface of the test element to be in extensive contact with the sampling face(s) of the sampling element and in this respect to allow fluidic communication between the two elements.

In a preferred variant of the method according to the invention, bringing the sampling element into contact with the test element produces a direct contact between the sample (or the analyte) and the analyte-binding receptor molecule on the test element. This produces a rapid contact between the analyte and the analyte-binding receptor molecule, as a result of which the complex of analyte and analyte-binding receptor molecule can rapidly form and the sensitivity and specificity of the detection method is significantly improved. Nevertheless, the present invention also provides the possibility that contact between the analyte and the analyte-binding receptor molecule only takes place after the sample has been transferred from the sampling element to the test element and only after chromatography has started.

To further improve the sensitivity and specificity of the method, in a preferred variant the test element also comprises at least one means which effects a chemical or/and mechanical treatment of the analyte-containing sample, for example by blocking or destroying non-specific binding sites in the sample or/and by changing the consistency of the sample. In this manner, the analyte is optimally available for binding to the analyte-binding receptor molecule, whereby the accessibility of the analyte to the analyte-binding receptor molecule and the transport over the individual regions of the test element is improved.

If a chemical sample-treatment means is used, this can be impregnated on the test element, for example, an aqueous or non-aqueous solution containing the chemical sample-treatment means in a concentration of approximately 0.01 to approximately 5 wt.-% preferably being used for impregnation. Particularly preferably, at least one chemical sample treatment means and at least one mechanical sample-treatment means are used in parallel in the test element.

Chemical sample-treatment means which can be used within the scope of the method according to the invention comprise in particular acids, bases, buffers, organic solvents and detergents, wherein bases are particularly preferred. Specific examples of acids comprise inorganic acids (for example hydrochloric acid) and organic acids (for example acetic acid and citric acid). Examples of bases comprise in particular alkali and alkaline earth metal hydroxides, such as sodium hydroxide and calcium hydroxide, while the buffers used can be, inter alia, calcium carbonate, Tris, PBS, phosphate buffer, borate buffer, BICINE buffer and HEPES. Examples of detergents comprise, inter alia, octyl glucoside, cholamido propanesulfonate, polidocanol, polyalkylene glycol ether (for example Brij®, Synperonic®) and polysorbates (for example Tween® 20, Tween® 80). Examples of organic solvents comprise in particular dimethyl sulfoxide, ethanol, glycerine, isopropanol, methanol and mixtures thereof.

Mechanical sample-treatment means, which can be used within the scope of the method according to the invention, comprise for example wovens or/and nonwovens, in particular nonwovens, using which the sample is filtered and separated before incubation of the analyte with the analyte-binding receptor molecule, so that solid and viscous sample constituents (for example solid and viscous saliva constituents) which can adversely affect the detection method are specifically retained. Specific examples of nonwovens which can bring about a mechanical treatment of the sample include for example the commercially available products Ahlstrom 8964, Whatman Rapid 24Q and Freudenberg FS 2216, but are not restricted thereto.

To make it easier for the user to bring the test element into contact with the sampling element, in an embodiment of the invention one or more test elements can be accommodated in a housing. The housing preferably has at least one opening via which the sampling element can be brought into contact with the test element. If the sampling element has a plurality of sampling faces, it is considered preferable for the housing to have only a single opening for receiving the sampling element. However alternatively, it is also possible for the housing to contain a number of openings which correspond to the number of sampling faces. The opening(s) can be arranged in any form (relative to one another) and can have any dimension and shape deemed suitable by a person skilled in the art, but it is/they are usually adapted to the arrangement, dimension and shape of the sampling face(s) of the sampling element. This measure makes it possible, for example, to obtain a previously defined joining of sampling element and test element to thus prevent incorrect use of the test system.

In a further embodiment, the housing can also comprise a retaining device which allows a reversible attachment of the sampling element to the housing, so that for example said sampling element can be removed for sampling and afterwards can be reattached to the housing. Here, by selecting a suitable position for the retaining device, the sampling element can be placed on the housing such that an intensive contact is ensured between sampling element or the sampling face(s) thereof and the test element, which ensures an effective transfer of the analyte from the sampling element to the test element comprising the analyte-binding receptor molecule.

Within the scope of the present invention, the test element and the sampling element, wetted with analyte, are preferably brought into contact with one another for a period of at least 10 seconds for the purpose of a high sensitivity and specificity of the analyte determination, wherein the analyte-binding receptor molecule on the test element can be incubated with the analyte to be determined and, if appropriate, the sample containing the analyte also is treated chemically or/and mechanically. This ensures that on the one hand the analyte is released as completely as possible from the sample matrix of the sampling element, and on the other hand it can react almost quantitatively with the analyte-binding receptor molecule. In this connection, an incubation time of from approximately 10 seconds to approximately 600 seconds, more preferably from approximately 20 seconds to approximately 180 seconds, most preferably from approximately 30 seconds to approximately 90 seconds, has proved to be advantageous. Due to a method of this type, the sample volume required for determining the analyte can usually be reduced to less than 10 μl and the sensitivity of the test system can be significantly improved.

After the analyte-containing sample has been applied onto the test element, the test element is brought into contact with an eluent. For this purpose, the test element preferably comprises an (end) region which is configured for receiving eluent and usually comprises an absorbent material, such as woven fabric or/and nonwoven fabric. After this region has been wetted with eluent, said eluent travels through the different regions of the test element, wherein the analyte, analyte-binding receptor molecule and the complexes thereof are transported along accordingly. Here, the capillary effect of the individual components of the test element can be advantageously made use of, which components are arranged or interconnected such that an uninterrupted flow of eluent is ensured.

Within the scope of the present invention, in principle any eluent deemed suitable by a person skilled in the art can be used as eluent. However, in the method described herein, water and aqueous buffer solutions are preferably used which can optionally comprise further substances such as a carbohydrate, a sugar alcohol, a detergent, a salt or/and an organic solvent, as respectively described above, in a concentration of usually approximately 0.05 to approximately 1.5 wt.-%. Particularly preferably, within the scope of the method according to the invention, an aqueous eluent is used which comprises as constituents magnesium sulphate and a combination of borates (for example magnesium-chlorine-borate, sodium tetraborate, calcium borate and calcium-sodium-borate), using which it is possible to achieve an increase in sensitivity or/and specificity of the analyte determination.

If the test element is introduced into a housing, there are various possibilities of wetting the test element with eluent, depending on the configuration of the housing. If the test element is mounted completely in the housing, the eluent can be applied onto the region of the test element configured for receiving eluent, for example by pressing onto an ampoule which contains the eluent and is preferably mounted inside the housing. However, if the region of the test element configured for receiving eluent projects out of the housing, it is possible to dip the region into the eluent.

After the test element has been brought into contact with the eluent, the determination of the analyte is started which can comprise, for example, a competitive test format or/and a non-competitive test format (sandwich test format). A combination of a competitive test format and a non-competitive test format is particularly preferred according to the invention. The detection method according to the invention usually initially comprises the formation of a complex of analyte molecules and analyte-binding, optionally labeled receptor molecule which is further transported by the eluent, for example together with non-bound receptor molecules or/and further substances present in the sample, as far as a region of the test element configured to detect the analyte.

The detection region, configured in particular for optically detecting the analyte, usually comprises a plurality of defined portions in which different reagents can be immobilised. In an embodiment, this region comprises a portion configured for binding non-bound analyte-binding receptor molecules, a portion configured for binding the complex consisting of analyte and analyte-binding receptor molecule, and optionally a portion in which a control signal is generated independently of the analyte. The analyte detection region can be formed from one or more materials deemed suitable for the purposes of the invention by a person skilled in the art, such as membranes consisting of Nylon®, nitrocellulose or polyvinylidene fluoride.

In the competitive test format, the portion provided for binding non-bound analyte-binding receptor molecules can comprise, for example, immobilised analyte analogues, in particular polyhaptens, which intercept the analyte-binding, optionally labeled receptor molecules due to the formation of a complex at a defined position on the test element (interception line) and in this way avoid the creation of false-positive results. The complex of analyte and analyte-binding receptor molecule is not usually immobilised at the interception line because the analyte blocks the binding sites, required for this, at the analyte-binding receptor molecule.

The portion configured for binding the complex of analyte and analyte-binding receptor molecule preferably comprises a binding partner which is specific to the analyte-binding receptor molecule (for example an antibody) which causes an immobilisation of the complex of analyte and analyte-binding receptor molecule at a predetermined position on the test element (test line) and allows an optical determination of the analyte if the analyte-binding receptor molecule bears a visual label. The complex is usually immobilised via a free binding site of the analyte-binding receptor molecule, accompanied by a colouring of the test line upon a positive detection of the analyte.

In order to be able to clearly read the signal at the test line and to rule out any confusion with the interception line, the interception line can optionally be covered in a suitable manner. Furthermore, if the test element comprises a control portion, when carrying out the method a control line also appears which acts as an indicator of fault-free functionality of the test element. Excess eluent which leaves the analyte detection region of the test element can optionally be absorbed by a liquid-absorbing material in a region of the test element specifically configured therefor.

In the non-competitive test format or sandwich test format, the analyte detection region does not comprise a portion configured for binding non-bound analyte-binding receptor molecule. In this case, to produce the complex of analyte and analyte-binding receptor molecule, a special analyte-specific binding partner is preferably used, as described for example in EP 1 579 222 B1. Consequently, complexes of this type can be immobilised by a complex-specific binding partner, which ultimately results in a simplified detection of the analyte, because the generation of false-positive signals which are caused, inter alia, by analyte-binding receptor molecules not bound to the interception line, are avoided.

The portion configured for binding the complex of analyte and analyte-binding receptor molecule then preferably comprises a complex-specific binding partner (for example an antibody) which causes an immobilisation of the complex of analyte and analyte-binding receptor molecule at a predetermined position on the test element (test line) and in this manner allows an optical determination of the analyte if the analyte-binding receptor molecule bears a visual label. The complex is usually immobilised via a free binding site of the complex-specific binding partner, accompanied by a colouring of the test line upon a positive detection of the analyte.

The qualitative or/and quantitative determination of the analyte, carried out in the last step of the method according to the invention, can be performed in any manner. For this, in principle, all methods known from the prior art for detecting a complex-formation can be used which generate a measurable signal which can be evaluated and read out manually or using suitable means. Within the scope of the present invention, optical detection methods are preferably used, in particular photometric or fluorimetric detection methods. An optical-visual detection of the analyte is particularly preferred according to the invention.

Particularly preferably, several of different analytes, in particular from 5 to 50 different analytes, are simultaneously determined by the method according to the invention. In this case, it is preferred that several different analytes from one substance class are simultaneously detected, for example different cannabinoids, by a single analyte-binding receptor molecule. If analytes from different substance classes are to be detected by the test element, the region configured for applying the sample can comprise in particular a number of different analyte-binding receptor molecules which corresponds to the number of different substance classes and, if the test element does not contain a portion for intercepting non-bound analyte-binding receptor molecules, it can optionally comprise a number of complex-specific binding partners which corresponds to the number of different substance classes, as defined above. This can ensure that analytes from different substance classes are determined in parallel substantially independently of one another and no interference occurs.

The method according to the invention allows the determination of one or more analytes with a high sensitivity and specificity. Thus, according to the invention it is preferred to determine analytes with a specificity of at least 95% or/and with a sensitivity of at least 90%. More preferably, analytes are determined with a specificity of at least 98% or/and with a sensitivity of at least 95%, so that by the method described herein, it is possible to detect analytes as far as a lower detection limit of approximately 1 ng/ml sample. According to the SAMHSA (Department of Health and Human Services: Proposed revisions to mandatory guidelines for federal workplace drug testing programs, Federal Register (2004), 69, 19673-19732) and Bosker et al. (Clin. Chem. (2009), 55, 1910-1931), the lower detection limit for Δ⁹-tetrahydrocannabinol should be 4 ng/ml saliva and the lower detection limit for synthetic cannabinoids should be 10 ng/ml saliva.

The method according to the invention can be used to determine any biological or chemical substance which is detectable, for example using immunological techniques or receptor-ligand techniques. However, the method described here is preferably used to detect natural, semi-synthetic or fully synthetic narcotics, as listed by the German narcotics law and which bind in vivo to a receptor molecule used according to the invention. Specific examples of controlled substances of this type include, inter alia, dissociatives, deliriants, empathogens, entactogens, hypnotics, narcotics, psychedelics, sedatives and stimulants, however without being restricted thereto.

More preferably, according to the invention at least one analyte selected from the group consisting of natural, semi-synthetic or fully synthetic amphetamines, benzodiazepines, cannabinoids, ketamines, methamphetamines, opioids and tropane alkaloids is determined, cannabinoids and opioids being preferred as analytes. Within the group of cannabinoids, Δ⁹-tetrahydrocannabinol and synthetic cannabinoids are preferred as analytes, while preferred examples of opioids include codeine, desomorphine, fentanyl, heroin, methadone and morphine. Particularly preferably, Δ⁹-tetrahydrocannabinol and synthetic cannabinoids are detected according to the invention.

The analyte can originate from any source, for example from an object wetted with the analyte, in particular from the surface of an object wetted with the analyte, or from a body fluid, in particular from blood, urine, saliva or sweat. The presence or/and quantity of an analyte in a sample of saliva or sweat is preferably determined by the method described herein. The amount of sample required for carrying out the method is usually from approximately 0.1 μl to approximately 200 μl, preferably from approximately 0.5 μl to approximately 40 μl, more preferably from approximately 1 μl to 15 μl and most preferably from approximately 2 μl to approximately 10 μl.

In a further aspect, the invention relates to a test element for determining an analyte, comprising:

(i) optionally a first region configured for absorbing eluent,

(ii) a second region configured for applying a sample containing the analyte,

(iii) a third region configured for detection, preferably for optically detecting the analyte, (iv) optionally a fourth region configured for absorbing excess eluent, and

(v) optionally a housing,

wherein the test element comprises at least one receptor molecule binding the analyte,

wherein the ligand-binding domain of the receptor molecule is present in native, shortened or mutated form and optionally is conjugated with a heterologous molecule.

In a further aspect, the invention relates to a sampling element for taking up an analyte from an object and transferring the analyte onto a test element, the sampling element comprising at least one receptor molecule binding the analyte, wherein the ligand-binding domain of the receptor molecule is present in native, shortened or mutated form and optionally is conjugated with a heterologous molecule.

In a further aspect, the invention relates to a kit for determining an analyte, which kit is preferably used for carrying out the method described above and comprises the following components:

(a) a test element, comprising
(i) optionally a first region configured for absorbing eluent,
(ii) a second region configured for applying a sample containing the analyte,
(iii) a third region configured for detection, preferably for optically detecting the analyte,
(iv) optionally a fourth region configured for absorbing excess eluent, and
(v) optionally a housing, and

(b) a sampling element configured for taking up a sample containing the analyte from a surface,

wherein the test element or/and the sampling element comprises at least one receptor molecule binding the analyte, wherein the ligand-binding domain of the receptor molecule is present in native, shortened or mutated form and optionally is conjugated with a heterologous molecule.

With regard to preferred configurations of the test element according to the invention, of the sampling element according to the invention and of the test element or sampling element contained in the kit according to the invention, reference is made to the embodiments in connection with the description of the method according to the invention.

### DESCRIPTION OF THE FIGURES

FIG. 1: shows the chemical structure of Δ⁹-tetrahydrocannabinol and of different selected structures from the group of synthetic cannabinoids.

FIG. 2: shows the chemical structure of different selected structures from the group of opioids.

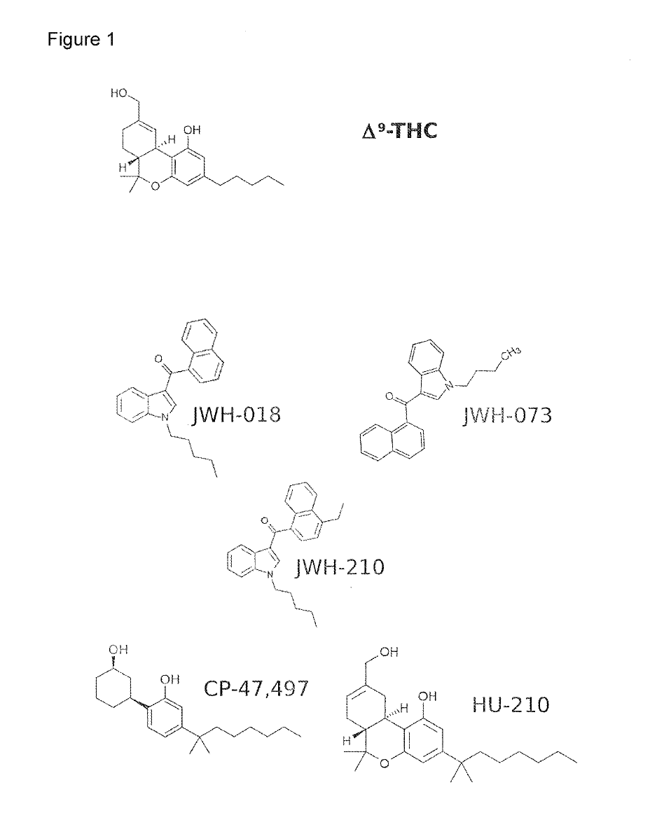

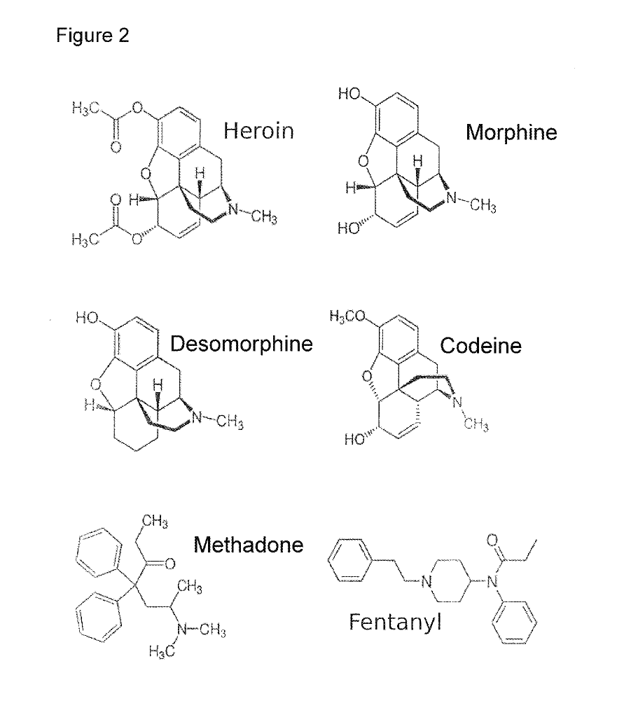

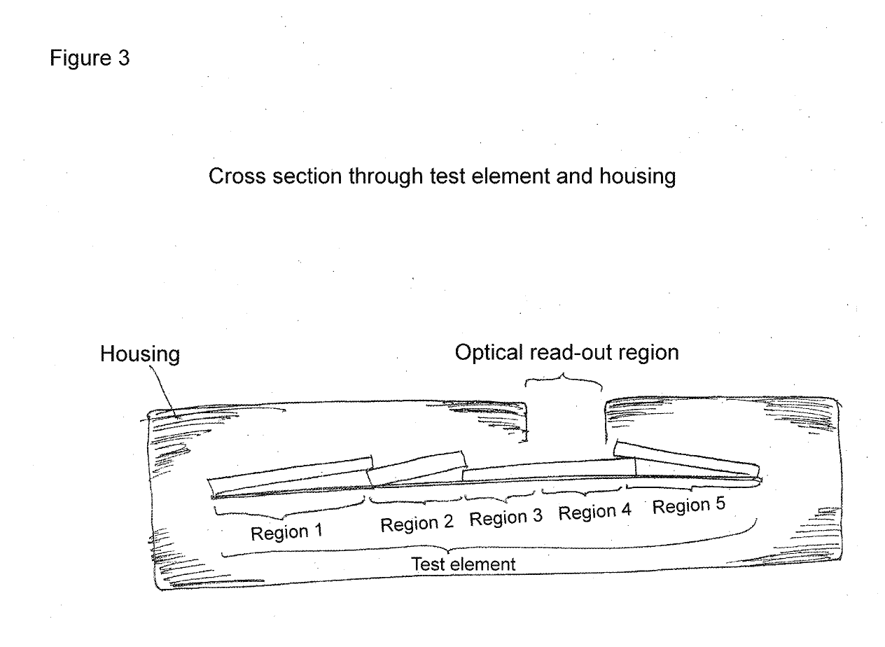

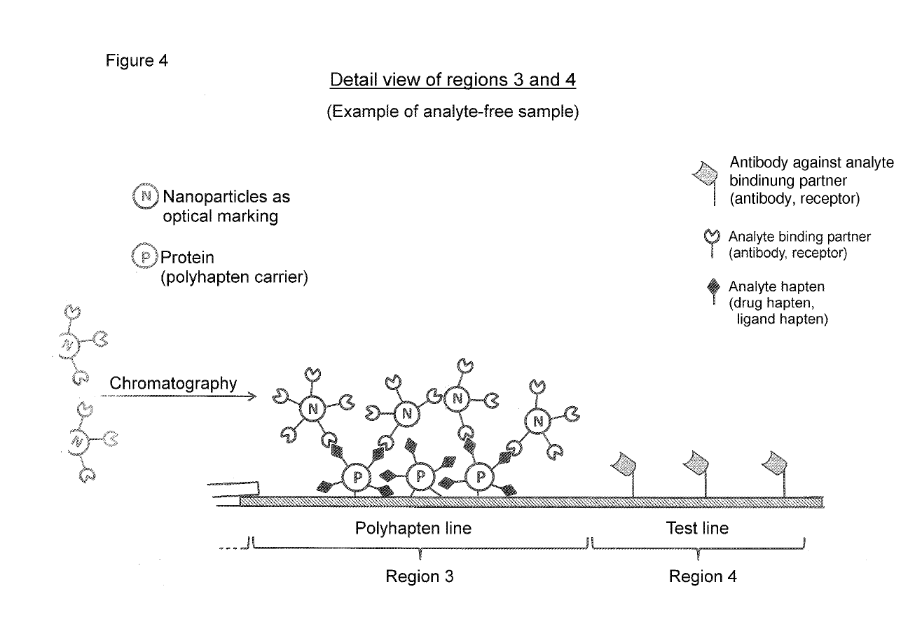

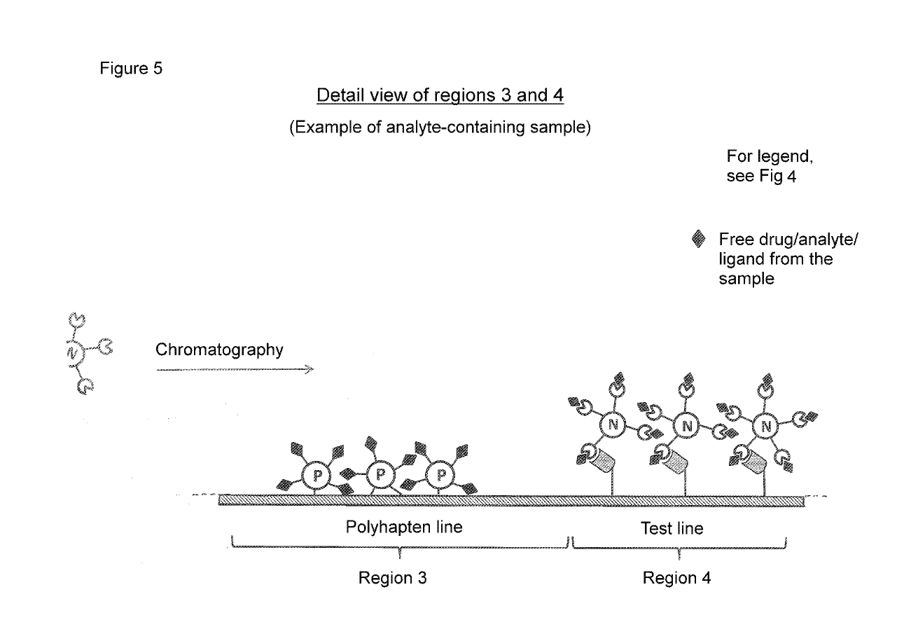

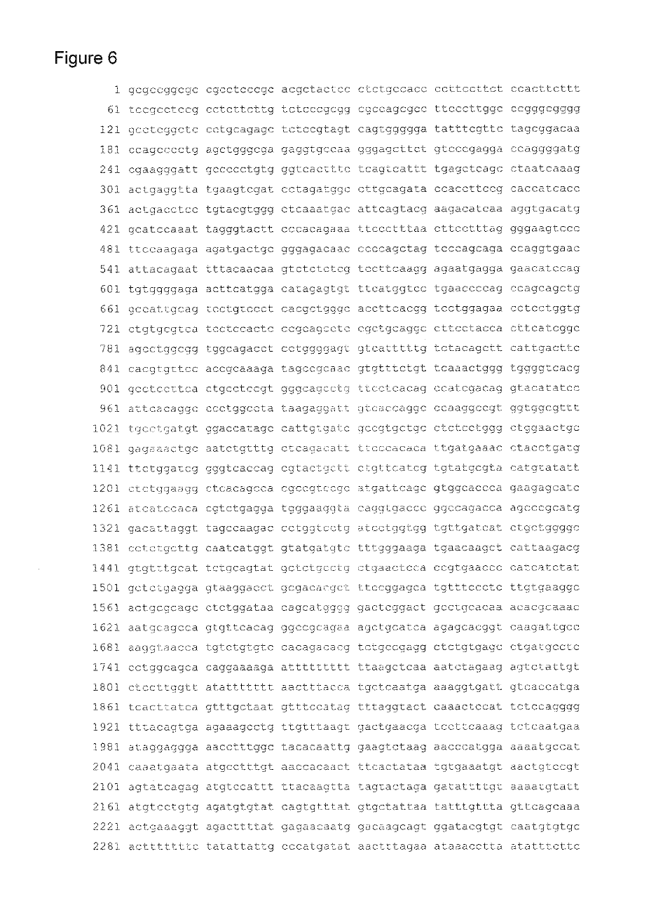

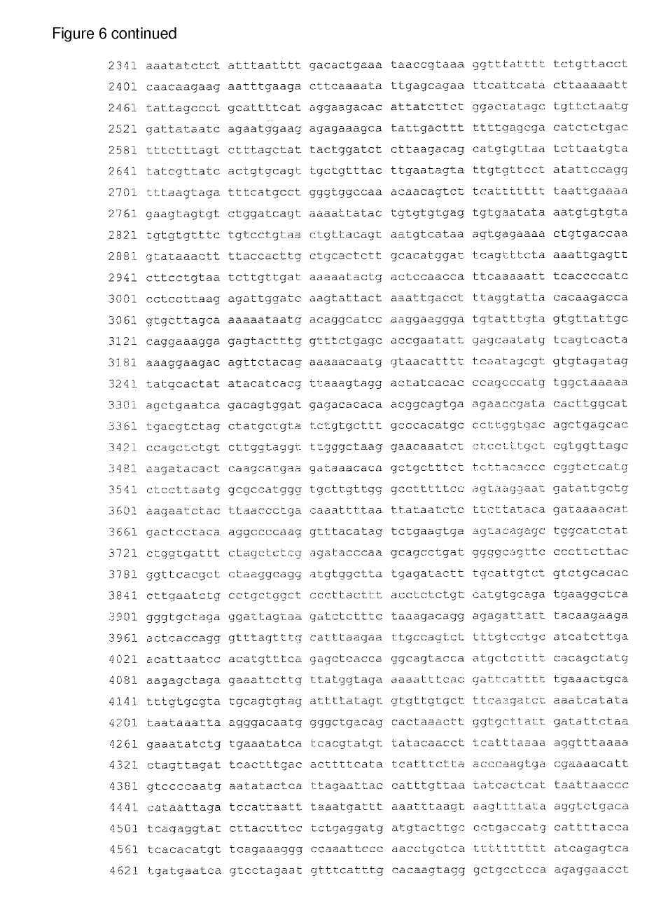

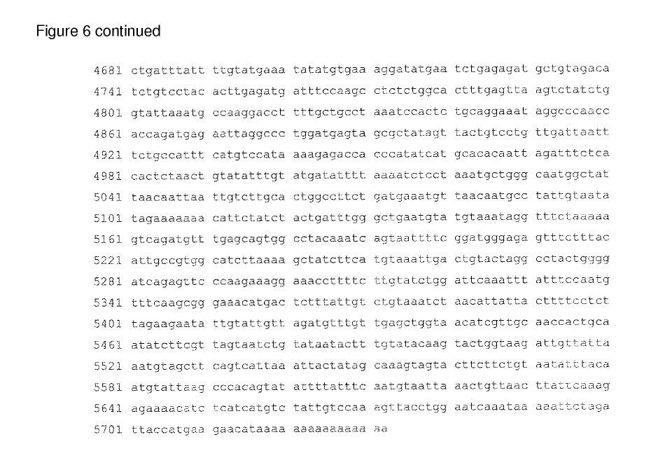

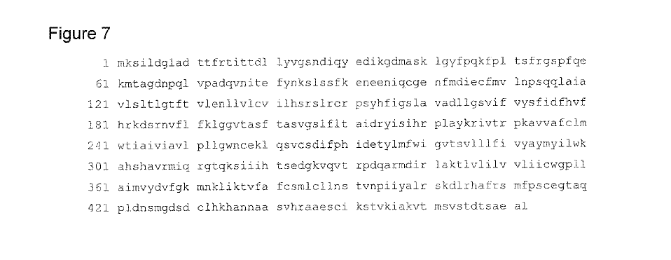

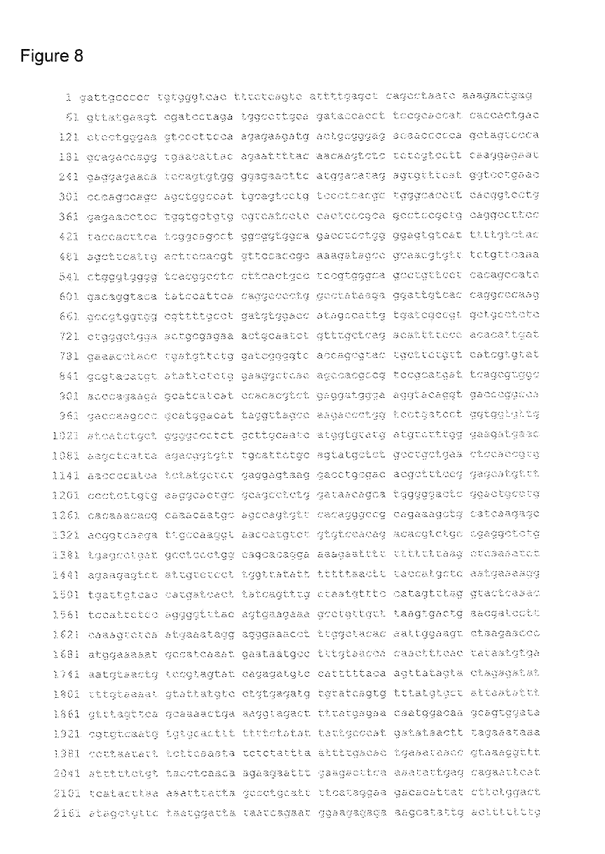

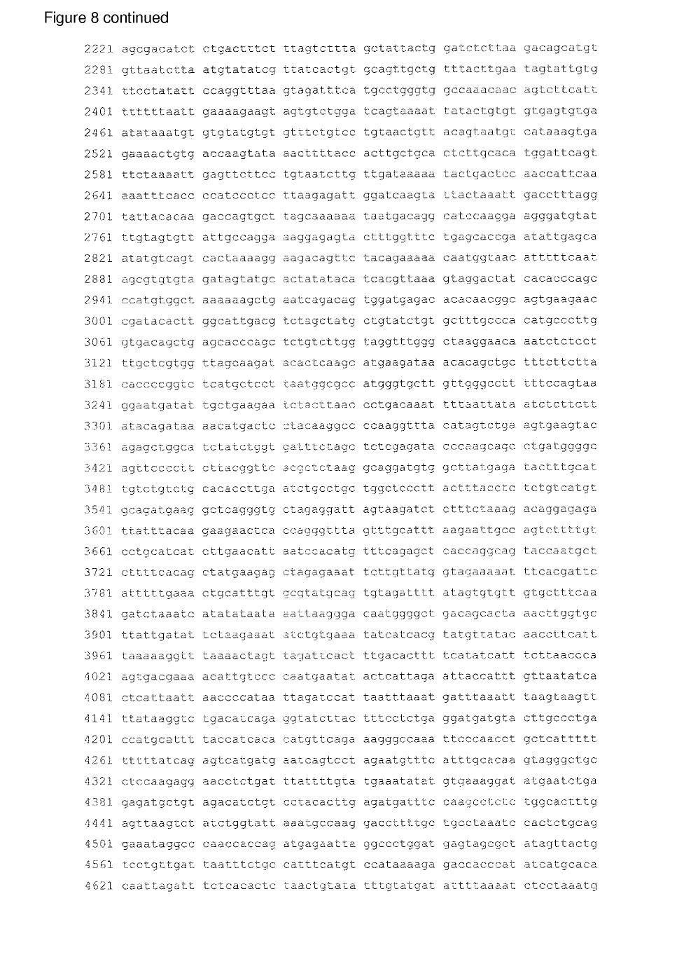

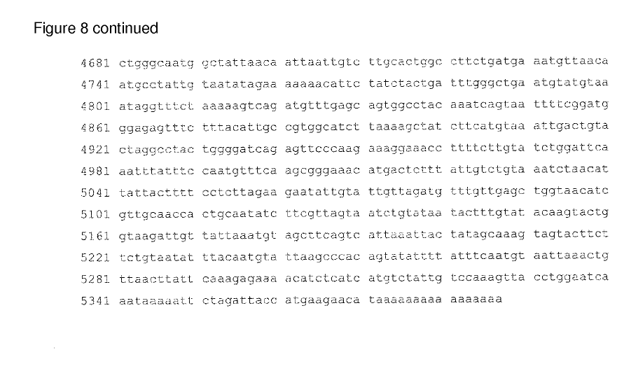

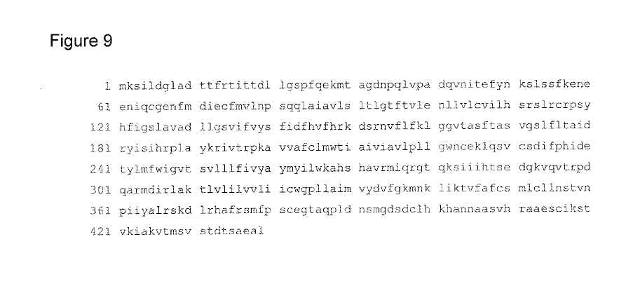

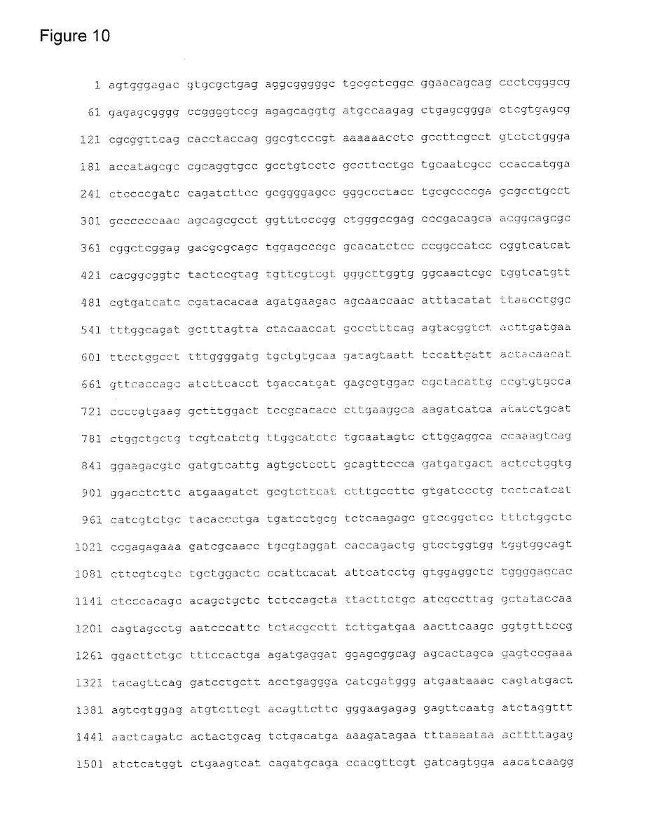

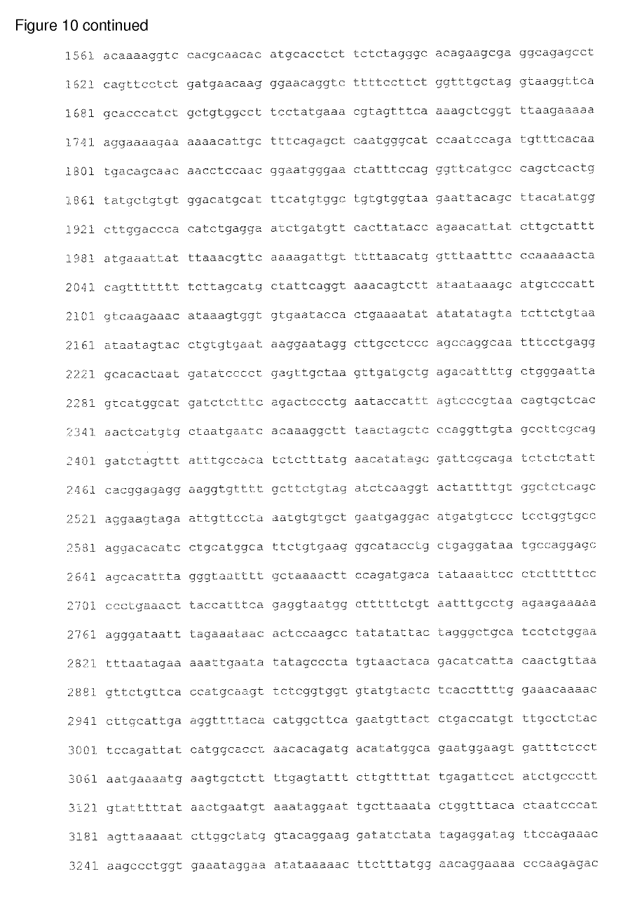

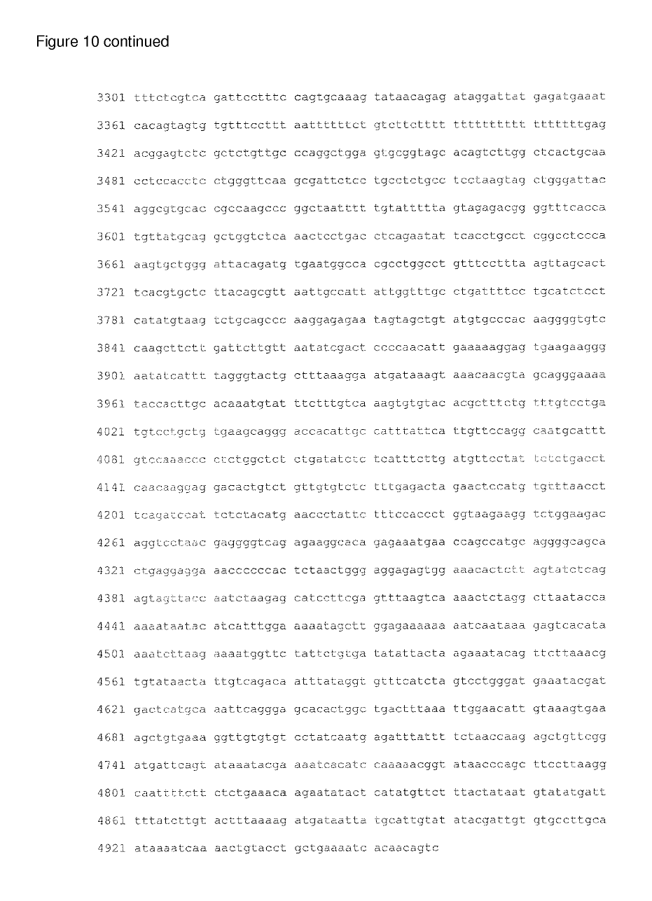

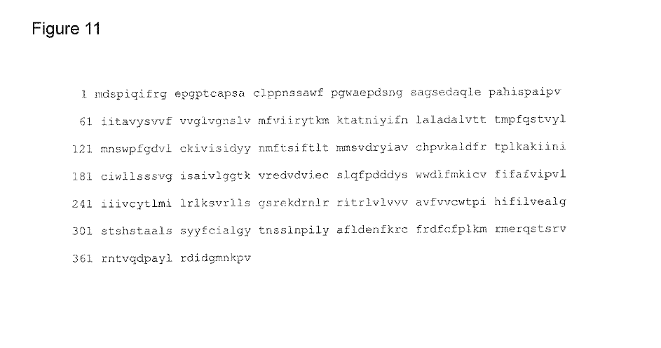

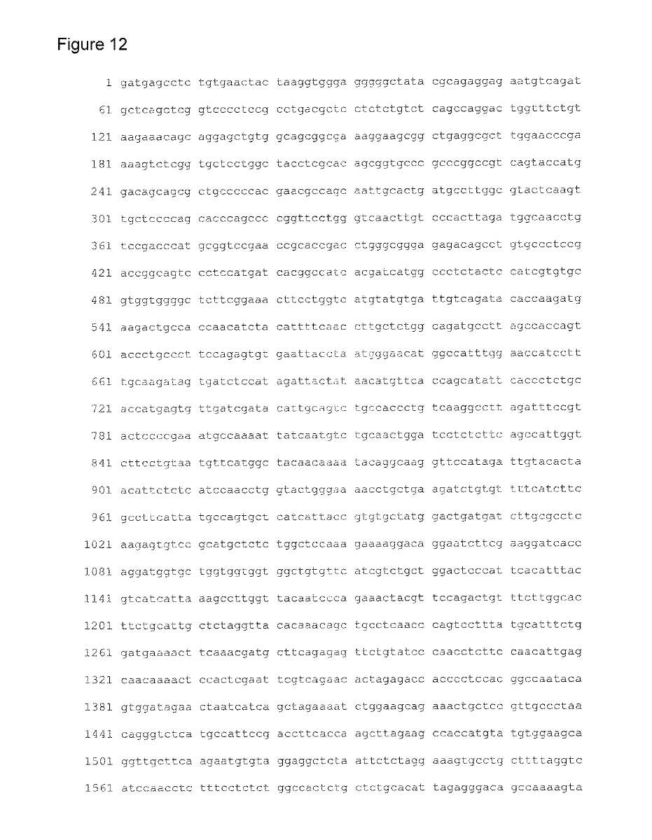

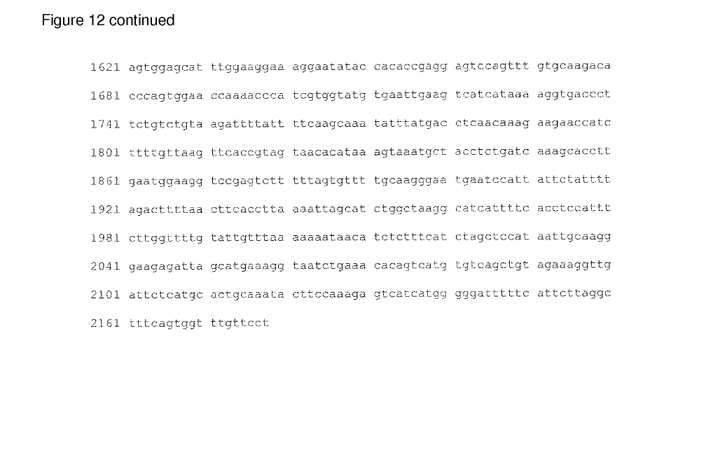

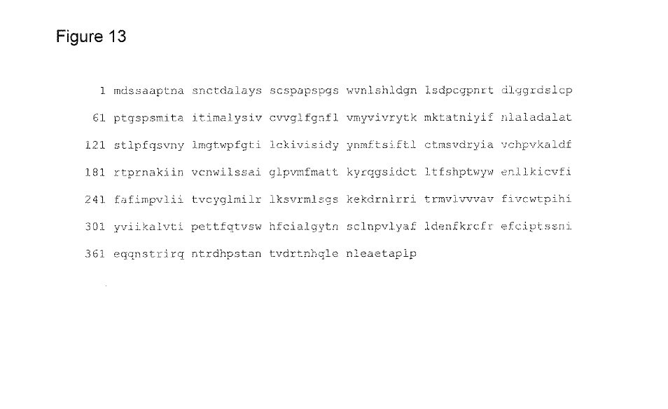

FIG. 3: is a cross-sectional view of an embodiment of a test element for carrying out the method according to the present invention.
The test element comprises:
(a) a first region configured for absorbing eluent,
(b) a second region configured for applying a sample containing the analyte and containing an analyte-binding receptor molecule and an analyte transfer reagent,
(c) a third region configured for binding non-bound analyte-binding receptor molecule and comprising polyhaptens,
(d) a fourth region configured for optically detecting the analyte and comprising a test line if a complex of analyte and analyte-binding receptor molecule has been formed,
(e) a fifth region configured for absorbing excess eluent, and
(f) a housing.

FIG. 4: is a detail view of regions 3 and 4 of the test element according to FIG. 3 in which the chromatography course of a sample without analyte is shown. The analyte-binding receptor molecule which is bound to the surface of suitable nanoparticles is immobilised at the polyhapten line in region 3 of the test element, since it interacts with the polyhaptens via its free binding sites.

FIG. 5: is a detail view of regions 3 and 4 of the test element according to FIG. 3 in which the chromatography course of a sample with analyte is shown. The analyte-binding receptor molecule which is bound to the surface of suitable nanoparticles is not immobilised at the polyhapten line in region 3 of the test element, since its binding sites are occupied by the analyte and interaction with the polyhaptens is impossible. Instead, the complex of analyte and analyte-binding receptor molecule travels further as far as region 4 of the test element where it is immobilised on the test line.

FIG. 6: shows the nucleotide sequence of human cannabinoid receptor 1 (SEQ ID NO:1).

FIG. 7: shows the amino acid sequence of human cannabinoid receptor 1 (SEQ ID NO:2).

FIG. 8: shows the nucleotide sequence of human cannabinoid receptor 2 (SEQ ID NO:3).

FIG. 9: shows the amino acid sequence of human cannabinoid receptor 2 (SEQ ID NO:4).

FIG. 10: shows the nucleotide sequence of human opioid receptor κ1 (SEQ ID NO:5).

FIG. 11: shows the amino acid sequence of human opioid receptor κ1 (SEQ ID NO:6).

FIG. 12: shows the nucleotide sequence of human opioid receptor μ1, transcript variant MOR-1 (SEQ ID NO:7).

FIG. 13: shows the amino acid sequence of human opioid receptor μ1, transcript variant MOR-1 (SEQ ID NO:8).

### EXAMPLE 1

### Use of Human Cannabinoid Receptor

To produce the human cannabinoid receptors 1 and 2 (SEQ IDs NO: 1 and NO:3 according to FIGS. 6 and 8) in a soluble and stable form, the methods described in Klammt et al. 2007 (Cell-free production of G protein-coupled receptors for functional and structural studies, J Struct Biol. 158(3):482-93), Schwarz et al. 2007 (Preparative scale cell-free expression systems: new tools for the large scale preparation of integral membrane proteins for functional and structural studies, Methods 41(4):355-69) and Ishiharaa et al. 2005 (Expression of G protein coupled receptors in a cell-free translational system using detergents and thioredoxin-fusion vectors, Protein Expression and Purification 41:27-37) are used.

The sequence-optimised cDNA sequences of human cannabinoid receptors 1 and 2 (SEQ IDs NO: 1 and NO: 3 from FIGS. 6 and 8) are cloned into a plasmid vector, type pET (pET21a(+) or pET100/D). The receptors were expressed in a cell-free environment and were purified as described in Klammt et al. 2004 (High level cell-free expression and specific labeling of integral membrane proteins, Eur. J. Biochem. 271(3):568-80) and Klammt et al. 2005 (Evaluation of detergents for the soluble expression of alpha-helical and beta-barrel-type integral membrane proteins by a preparative scale individual cell-free expression systems, FEBS J. 272(23):6024-38).

The two purified cannabinoid receptors 1 and 2 are conjugated together or individually in each case on the surface of 40 nm gold particles. For conjugation, a di-sodium tetraborate buffer is used to achieve optimum binding to the gold surface.

Using a gold conjugate dispenser manufactured by BioDot, the gold conjugate is introduced onto the nonwoven fabric of a lateral flow test strip and then dried. The nonwoven fabric impregnated with gold conjugate is stored at a relative air humidity of <5%.

By adding mobile solvent (for example a running buffer), the dried-in gold conjugate is dissolved and travels over the test strip. Located on the test strip is a zone which contains proteins, on the surface of which Δ⁹ THC molecules have been applied. The gold conjugate binds to these immobilised Δ⁹ THC molecules if there is no Δ⁹ THC or no other cannabinoid in a sample to be examined. However, if Δ⁹ THC or another cannabinoid is present in the sample, it binds to the cannabinoid receptors on the surface of the gold conjugate. Consequently, the cannabinoid receptors cannot then bind to the immobilised Δ⁹ THC molecules on the test strip. Thus, this rapid lateral flow test can provide a Yes/No indication about the presence or absence of cannabinoids in a sample.

### EXAMPLE 2

### Use of Human Opioid Receptor

For a successful expression of opioid receptor μ (SEQ ID NO:7, FIG. 12) in soluble form, a sequence optimisation can be carried out as described in Maertens et al. 2010 (Gene optimization mechanisms: A multi-gene study reveals a high success rate of full-length human proteins expressed in Escherichia coli, Protein Science 19:1312-1326), Corin et al. 2011 (A Robust and Rapid Method of Producing Soluble, Stable, and Functional G-Protein Coupled Receptors, PLoS ONE 6 (10) e23036), Koth & Payandeh 2009 (Strategies for the cloning and expression of membrane proteins, Adv Protein Chem. Struct. Biol. 76:43-86) and Link et al. 2008 (Efficient production of membrane-integrated and detergent-soluble G protein-coupled receptors in Escherichia coli, Protein Science 17:1857-1863).

Furthermore, for a water-soluble variant of the coupled opioid-receptor μ a computer-assisted design of the protein can be used (Perez-Aguilar et al. 2013: A Computationally Designed Water-Soluble Variant of a G-Protein-Coupled Receptor: The Human Mu Opioid Receptor, PLoS ONE 8(6) e66009).

The sequence-optimised cDNA sequence of the human opioid receptor μ is cloned into the expression plasmid pET-28b(+) (EMD/Novagen) which is transformed into the bacterial strain E. coli BL21(DE3) (EMD/Novagen) to express the receptor. Expression and purification are carried out as described in Perez-Aguilar et al. 2013: (A Computationally Designed Water-Soluble Variant of a G-Protein-Coupled Receptor: The Human Mu Opioid Receptor, PLoS ONE 8(6) e66009).

The purified opioid receptor μ is conjugated onto the surface of 40 nm gold particles. For conjugation, a di-sodium tetraborate buffer is used to achieve optimum binding.

Using a gold conjugate dispenser manufactured by BioDot, the gold conjugate is introduced onto the nonwoven fabric of a lateral flow test strip and then dried. The nonwoven fabric impregnated with gold conjugate is stored at a relative air humidity of <5%.

By adding mobile solvent (for example a running buffer), the dried-in gold conjugate is dissolved and travels over the test strip. Located on the test strip is a zone which contains proteins, on the surface of which morphine molecules have been applied. The gold conjugate binds to these immobilised morphine molecules if there is no morphine in a sample to be examined. However, if an opioid is present in the sample, it binds to the opioid receptors on the surface of the gold conjugate. Consequently, the opioid receptors can no longer bind to the immobilised morphine molecules on the test strip. Thus, this rapid lateral flow test can provide a Yes/No indication about the presence or absence of opioids in a sample.

1. Method for determining an analyte in a sample, comprising the steps of:
(a) providing a test element,
(b) applying the sample to the test element, and
(c) determining the presence or/and quantity of analyte on the test element, characterised in that
the test element comprises at least one receptor molecule binding the analyte,
wherein the receptor molecule comprises the ligand-binding domain of a narcotic-binding, G protein-coupled receptor molecule, in particular the ligand-binding domain of a cannabinoid-binding or opioid-binding receptor molecule, and
wherein the ligand-binding domain of the receptor molecule is present in a native, shortened or mutated form and optionally is conjugated with a heterologous molecule.

2. Method for determining an analyte in a sample, comprising the steps of:
(a) providing a test element,
(b) taking up the sample from a surface using a sampling element, said sampling element having one or more sampling faces,
(c) bringing the sampling element into contact with the test element, at least a part of the sample being transferred from the sampling element onto the test element, and
(d) determining the presence or/and quantity of analyte transferred onto the test element,
characterised in that
the test element comprises at least one receptor molecule binding the analyte, wherein the receptor molecule comprises the ligand-binding domain of a narcotic-binding, G protein-coupled receptor molecule, in particular the ligand-binding domain of a cannabinoid-binding or opioid-binding receptor molecule, and
wherein the ligand-binding domain of the receptor molecule is present in a native, shortened or mutated form and optionally is conjugated with a heterologous molecule.

3. Method according to claim 1, characterised in that the receptor molecule is present in soluble form or is immobilised on the test element.

4. Method according to claim 1, characterised in that a test element is used for carrying out a heterogeneous, homogeneous or chromatographic test, an ELISA or a FRET test, wherein the test element optionally contains microfluidic structures and preferably is a chromatographic test strip.

5. Method according to claim 1, characterised in that the receptor molecule comprises the ligand-binding domain of a receptor molecule selected from the group consisting of cannabinoid receptor 1, cannabinoid receptor 2, opioid receptor δ, opioid receptor κ, opioid receptor μ1 and opioid receptor μ2.

6. Method according to claim 1, characterised in that the receptor molecule, in particular the ligand-binding domain thereof has a shortened amino acid sequence, in particular it has a shortening of the amino acid sequence by up to 10%, 20%, 30%, 40% or 50% compared to the native amino acid sequence, wherein the shortening is at the N terminus, at the C terminus or/and within the protein sequence.

7. Method according to claim 1, characterised in that the receptor molecule, in particular the ligand-binding domain thereof has a mutated amino acid sequence which is identical to at least 80%, in particular to at least 95%, to the amino acid sequence of the ligand-binding domain of the native receptor molecule.

8. Method according to claim 1, characterised in that the native receptor molecule originates from a mammal, in particular from a human being.

9. Method according to claim 1, characterised in that the receptor molecule is conjugated with a heterologous polypeptide, in particular with an immunoglobulin domain.

10. Method according to claim 1, characterised in that the sample
(a) is a body fluid, in particular blood, urine, saliva or sweat, or
(b) is taken from an object, in particular from the surface of an object.

11. Method according to claim 1, characterised in that the analyte is a natural, semi-synthetic or fully synthetic narcotic, in particular a cannabinoid or opioid which binds in vivo to the receptor molecule.

12. Method according to claim 1, characterised in that several analytes, in particular 5 to 50 different analytes are determined simultaneously, in particular several different analytes being detected together via a single receptor molecule.

13. Method according to claim 1, characterised in that it comprises a combination of a competitive test format and a non-competitive test format.

14. Test element for determining an analyte, comprising:
(i) optionally a first region configured for absorbing eluent,
(ii) a second region configured for applying a sample containing the analyte,
(iii) a third region configured for detection, preferably for optically detecting the analyte,
(iv) optionally a fourth region configured for absorbing excess eluent, and
(v) optionally a housing,
characterised in that
it comprises at least one receptor molecule binding the analyte,
wherein the receptor molecule comprises the ligand-binding domain of a narcotic-binding, G protein-coupled receptor molecule, in particular the ligand-binding domain of a cannabinoid-binding or opioid-binding receptor molecule, and
wherein the ligand-binding domain of the receptor molecule is present in a native, shortened or mutated form and optionally is conjugated with a heterologous molecule.

15. Sampling element for taking up an analyte from an object and for transferring the analyte onto a test element,
characterised in that
it comprises at least one receptor molecule binding the analyte,
wherein the receptor molecule comprises the ligand-binding domain of a narcotic-binding, G protein-coupled receptor molecule, in particular the ligand-binding domain of a cannabinoid-binding or opioid-binding receptor molecule, and
wherein the ligand-binding domain of the receptor molecule is present in a native, shortened or mutated form and optionally is conjugated with a heterologous molecule.

16. Kit for determining an analyte, comprising:
(a) a test element, comprising
(i) optionally a first region configured for absorbing eluent,
(ii) a second region configured for applying a sample containing the analyte,
(iii) a third region configured for detection, preferably for optically detecting the analyte,
(iv) optionally a fourth region configured for absorbing excess eluent, and
(v) optionally a housing, and
(b) a sampling element configured for taking up a sample containing the analyte from a surface,
characterised in that
the test element or/and the sampling element comprises at least one receptor molecule binding the analyte, wherein the receptor molecule comprises the ligand-binding domain of a narcotic-binding, G protein-coupled receptor molecule, in particular the ligand-binding domain of a cannabinoid-binding or opioid-binding receptor molecule, and
wherein the ligand-binding domain of the receptor molecule is present in native, shortened or mutated form and optionally is conjugated with a heterologous molecule.
